(19) United States
(12) Patent Application Publication
(10) Pub. No.: US 2005/0181516 A1
(43) Pub. Date: Aug. 18, 2005

(54) Bioequivalence determination using expression profiling

(76) Inventors: Marlene Michelle Dressman, Germantown, MD (US); Christian Nicolas Lavedan, Potomac, MD (US); Lee Anne Mc Lean, Germantown, MD (US); Mihael Hristos Polymeropoulos, Potomac, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/512,709

(22) PCT Filed: Apr. 30, 2003
(86) PCT No.: PCT/EP03/04582

(30) Foreign Application Priority Data
May 2, 2002 (US) 60377446

(51) Int. Cl. A61K 38/00; A61K 31/335; G05D 7/00; G01N 30/02; G05D 11/00; G01N 33/543; A61K 9/22; A01N 43/02

(52) U.S. Cl. 436/161; 424/468; 436/518; 514/11; 514/449; 700/283

(57) ABSTRACT

The present invention provides methods to use expression profiles to determine the bioequivalence of two or more pharmaceutically equivalent drug formulations. In addition, this invention provides methods to select drug formulations that are able to substitute for standard drug formulations without change in clinical efficacy. In other embodiments, this invention provides computer systems, kits and databases for carrying out the methods of the invention.

### BACKGROUND OF THE INVENTION

1. Field of the Invention

This invention relates to the field of expression profiling systems and the use of expression profiling to perform in vivo function based determinations of bioequivalence of two or more therapeutic regimens in a subject, preferably a human patient. In particular, this invention relates to methods, kits, databases and computer systems for monitoring and comparing the bioequivalence of various treatment regimens in a subject and to use these comparisons to determine appropriate therapeutic choices.

2. Description of the Related Art

In recent years, advances in several technologies have made it possible to monitor the expression level of a large number of genetic transcripts within a cell at any one time, this is referred to as “gene expression profiling”. In organisms for which the complete genome is known it is possible to analyze the transcripts of all genes within the cell. In humans and other organisms for which there is an increasing knowledge of the genome, it is now possible to simultaneously monitor large numbers of the genes within the cell (see, e.g., Schena et al., Science, Vol. 270, pp. 467-470 (1995); Lockhart et al., Nat. Biotech., Vol. 14, pp. 1675-1680 (1996); Blanchard et al., Nat. Biotech., Vol. 14, p. 1649 (1996); Ashby et al., U.S. Pat. No. 5,569,588, issued Oct. 29, 1996) and proteins (see, e.g., McCormack et al., Anal. Chem., Vol. 69, pp. 767-776 (1997); Chait-BT, Nat. Biotech., Vol. 14, p. 1544 (1996)).

Applications of this technology have included, for example, identification of genes which are up regulated or down regulated in various physiological states, particularly diseased states. Additional uses for transcript arrays and comparisons of gene profiles have included the analyses of members of signaling pathways, the identification of targets for various drugs, the assessment of the state or severity of a specific disease and the monitoring of the effect of a therapeutic regimen in a disease state (see, e.g., U.S. Pat. Nos. 6,218,122; 5,965,352; 5,811,231; 6,190,857; 5,777,888; 6,190,857; 6,146,830; 5,800,992; 5,723,290; 5,695,937; 6,203,987; 5,741,666; 5,702,902; 5,695,937; 6,222,093; 5,769,074; 5,707,807; 5,741,666; 5,702,902; 5,935,060; 6,222,093; and 5,569,588 and U.S. Patent Application Publication No. U.S. 2001/0018182 A1 and PCT Publication Nos. WO 99/66024; WO 00/39338; WO 00/39337; WO 01/11082; WO 00/58520; WO 00/24936; WO 01/20043 A1; WO 00/58520, WO 99/57324; WO 01/34789; WO 99/58708 and WO 00/39336, all incorporated by reference herein in their entirety and for all purposes).

Such applications are based upon the knowledge that abundances and/or activity levels of cellular constituents such as messenger RNA (mRNA) species and including proteins, and other molecular species within a cell, change in response to virtually any alteration in a cell's biological state or environment. Such “alterations” may include, but are not limited to, specific drug treatments or regimens or subtle changes in the biologic or chemical environment of a cell or the cells of an organism. Such an alteration may result from changes in the bioavailability of a drug due to minor changes in formulation, nature of excipients, composition, crystal form or any physiochemical property of the drug substance itself or the drugs delivery system. Thus, a measurement of such a plurality of cellular constituents, referred to herein as an “expression profile” or a gene expression profile (when referring to the measurement of mRNAs), contains a wealth of information about the nature and effect of the alteration. In addition, such an expression profile would be uniquely determined by the specifics of the alteration and thus allow the isolation and comparison of any of the above factors.

The ability to measure and compare such biological profiles has the potential to be of great human and commercial benefit. For example, it would be of great benefit during the process of drug discovery and design of therapeutic regimens, to provide and compare response profiles of known or existing drugs to new candidate drugs. It would also be of great benefit to establish functionally based gene expression response profiles that relate to specific and identifiable aspects of the pharmacokinetic factors that determine the bioequivalence of a drug formulation or regimen. This would allow the identification of drug formulations with bioequivalence identical to that of a known standard drug and therefore allow the identification of drug candidates with a particular desired therapeutic effect that is identical to a known standard drug. In addition, this would help to develop theories of why particular individual compounds, formulations, combinations, compositions, excipients, etc. have varying bioequivalence and how this may relate to clinically relevant efficacy or toxicity characteristics.

Pharmacodynamics

Pharmacodynamics, as opposed to pharmacokinetics, is the study of the biochemical and physiological effects of drugs and their mechanisms of action. The focus of this analysis is to delineate the chemical or physical interactions between the drug and the target cell. The effects of most drugs result from the interaction of the drug with the macromolecular components of the organism or cell. Usually these components are in the form of a receptor. The term “receptor” means any cellular macromolecule to which a drug binds to initiate its effects. Many of the most important drug receptors are cellular proteins whose normal function is to act as receptors for endogenous regulatory ligands particularly hormones, growth factors, neurotransmitters and autacoids. These receptors are often highly selective because these physiological receptors are specialized to recognize and respond to individuals signaling molecules with great selectivity.

Drugs which bind to receptors and mimic the effect of the endogenous regulatory compounds are termed “agonists”, those which inhibit the action of an agonist are termed “antagonists”. Agents which partly act as agonists are termed “partial agonists” and those agents that stabilize the receptor from undergoing productive agonist-dependent conformational changes are termed “negative antagonists” or “inverse agonists”.

The regulatory actions of a receptor may be exerted directly on its cellular targets, effector proteins or may be transmitted to cellular targets by intermediary cellular molecules or transducers. This often complex series of actions is termed the “receptor's signal transduction pathway”.

The ultimate end effect of the drug receptor interaction may be mediated by a wide variety of effector mechanisms. This mechanism may involve the phosphorylation or other alteration of a target protein such as an enzyme, a regulatory protein or a structural protein. The mechanism may involve a cytoplasmic second messenger such as cyclic AMP or calcium ion concentration. Another mechanism involves the regulation of ion-selective channels in the plasma membrane ligand-gated ion channels which convey their signals by altering the cell's membrane potential. One important type of effector mechanism is exemplified by receptors for, for example, steroid hormones, thyroid hormones, vitamin D and the retinoids—these are soluble DNA-binding proteins that act as transcription factors and regulate the transcription of specific genes (see Evans, Science, Vol. 240, pp. 889-895 (1988)).

Some drugs do not act by binding to receptors. Examples of such drugs are the use of mannitol to control the osmolarity of various body fluids or the use of structural analogs of normal biological chemical which may be incorporated into cellular components and thereby alter their function.

Pharmacokinetics

The term “pharmacokinetics” as used herein, as distinguished from pharmacodynamics, refers to all factors related to the dynamics of drug absorption, distribution in body tissues or fluids and metabolism and/or elimination. This involves the physicochemical factors that regulate the transfer of the drug across membranes because the absorption, distribution, biotransformation and excretion of a drug all involve the passage of the drug across cell membranes.

Of great interest to the clinician is the bioavailability of a drug. This term, as used herein, indicates the extent to which a drug reaches its site of action or a biological fluid from which the drug has access to its site of action. The factors affecting bioavailability include rate of absorption and metabolism or elimination of the drug from the subject. Many factors affect absorption, these include the numerous physicochemical factors that affect transport across membranes such as a drug solubility and uptake mechanisms as well as factors such as site of administration and the formulation and composition of the drug. The various routes of drug administration have markedly different absorption characteristics. These routes include oral ingestion, pulmonary absorption, parenteral injection, including; intramuscular, subcutaneous, intravenous, intraarterial, intrathecal or intraperitoneal injection and topical application to mucous membranes, skin or eye.

After a drug is absorbed or injected into the bloodstream, it may be distributed into interstitial and cellular fluids. The pattern of the distribution of a specific drug is a function of both the physicochemical properties of the drug and certain physiological factors of the host. These factors include blood flow, lipid solubility and protein binding characteristics of the drug as well as pH and the permeability of capillary endothelial membranes. This last factor is involved in determining one of the most clinically relevant distribution factors, the blood brain barrier. The endothelial cells of the brain-capillaries differ from their counterparts in most tissues by the absence of intercellular pores and pinocytotic vesicles. Instead, tight junction predominate and aqueous bulk flow is thus severely restricted. This limits the access of many drugs, especially non-lipid soluble ones, to the central nervous system (CNS) in mammals, including humans.

The distribution of a drug can also be affected by the tendency of the drug to accumulate or concentrate in specific tissues of the body. Thus, some drugs may accumulate in cells rather than in the extracellular fluid or may cross epithelial cells and accumulate in transcellular fluids or may preferentially concentrate in lipid (fat) or bone. The effect of a drug may also be altered by redistribution, for example, when a highly lipid-soluble CNS active drug is administered by intravenous (i.v.) injection but is rapidly take up by fat tissue.

The bioavailability of a drug is also determined by the biotransformation or metabolism. This can usefully be divided into Phase I and Phase II biotransformations. Phase I reactions are those which introduce or expose a functional group on the parent compound such as oxidation hydroxylation or deamination. Phase I reactions often result in loss of pharmacological activity and the altered drug may be rapidly excreted into the urine or may then react with endogenous compounds to form water soluble conjugates that are excreted in the urine.

Phase II conjugation reactions lead to the formation of a covalent linkage between a functional group on the parent compound and endogenous glucuronic acid, sulfate, glutathione, amino acids or acetate. The resulting, highly polar, conjugates are generally inactive and are excreted rapidly in the urine and feces.

The metabolic conversion of drugs is mostly enzymatic in nature and often takes place in the liver. However, other organs also have significant metabolic capacity including the kidneys, gastrointestinal tract, skin and lungs. One of the most important and complex enzyme systems is the cytochrome P450 monoxygenase system. This system consists of heme-containing membrane proteins localized in the smooth endoplasmic reticulum of numerous tissues. Examples of other important enzyme systems include those that catalyze hydrolytic and conjugation reactions.

Many other factors may affect the biotransformation of drugs. These factors include the induction or inhibition of enzyme systems, genetic polymorphisms, disease, age, gender and drug-drug interactions. Finally, the bioavailability of a drug is largely determined by the route and efficiency of its excretion and elimination from the body. The kidney is the most important organ for elimination of drugs and their metabolites. Excretion of drugs and metabolites in the urine involves three processes: glomerular filtration, active tubular secretion and passive tubular reabsorption. Many metabolites of drugs formed in the liver are excreted into the intestinal tract in the bile and either excreted in the feces or reabsorbed into the blood and ultimately excreted in the urine. Other drugs may be excreted into sweat, saliva, tears, hair or skin and breast milk (see, Goodman and Gilman's “The Pharmacological Basis of Therapeutics”, 9^{th} Ed., Hardman and Limbird, eds., McGraw-Hill, NY (1996), see especially Chapters 1, 2, 3 and Appendix 1).

Bioequivalence

The phenomenon of bioequivalence and the precise determination of the degree of bioequivalence between otherwise very similar pharmaceutical products has become one of the most important and difficult practical issues in the pharmaceutical industry (see, e.g., Welage, et al., J. Am. Pharm. Assoc., Vol. 41, No. 6, pp. 856-867 (2001); Senn, Stat. Med., Vol. 20, Nos. 17-18, pp. 2785-2799 (2001); Nerurkar et al., J. Clin. Pharmcol., Vol. 32, No. 10, pp. 935-943 (1992); Hendeles, Am. J. Hosp. Pharm., Vol. 50, No. 5, p. 913 (1993)). Drug products are considered to be “pharmaceutical equivalents”, as that term is used herein, if they contain the same active ingredients (chemically identical active drug substance or substances) and are identical in strength or concentration, dosage form and route of administration. Two pharmaceutically equivalent drug products are considered to be “bioequivalent”, as that term is used herein, when the rates and extents of bioavailability of the active ingredient(s) in the two products are not significantly different under suitable test conditions.

In the past, dosage forms of a drug from different manufacturers and even different lots of preparations from a single manufacturer sometimes differed in their bioavailability. Such differences were seen primarily among oral dosage forms of poorly soluble, slowly absorbed drugs. These variations result from differences in crystal form, particle size, or other physical characteristics of the drug that may not be rigidly controlled by some manufacturers in the formulation and manufacture of their products. These factors can affect, for example, the disintegration of the dosage form and dissolution of the drug and hence the rate and extent of drug absorption.

The potential variation in bioequivalence of different drug preparations is a matter of great concern to the pharmaceutical industry, to clinicians and, most of all, to patients. Strengthened regulatory requirements have been necessary to decrease the variation in bioequivalence between approved, ostensibly identical, drug products. However, the significance of possible non-bioequivalence of drug preparations is an enormous public health concern. Public policy dictates that necessary medications be made available at reasonable cost to the consumer and this cause can be furthered by the introduction of generic drugs to compete in the marketplace with brand name drugs.

However, despite the best efforts of the pharmaceutical industry and governmental regulatory agencies to ensure that a generic drug is only approved for sale if it is a bioequivalent formulation of the name brand drug, in practice this has not always been the case. Many physicians in clinical practice have found that when patients are switched from a name brand drug to a, possibly less expensive, generic that a clinically significant or even a serious or dangerous change in pharmacological effect results because of unanticipated variations in bioequivalence (see Ronald et al., Neurology, Vol. 57, pp. 571-573 (2001); Keith, Int. J. Fertil. Womens. Med., Vol. 46, No. 6, pp. 286-295 (2001); Balter et al., Clin. Ther., Vol. 23, No. 10, pp. 1720-1731 (2001); Lam, J. Clin. Psychiatry, Vol. 62, Suppl. 5, pp. 18-22 (2001); Wagner et al., Pharmacotherapy, Vol. 20, No. 2, pp. 240-243 (2000); Welty, Ann. Pharmacother., Vol. 26, No. 6, pp. 775-777 (1992); Nerurkar et al., J. Clin. Pharmacol., Vol. 32, No. 10, pp. 935-943 (1992); Lesser et al., Neurology, Vol. 57, pp. 571-573 (2001); Lam et al., J. Clin. Psychiatry, Vol. 62, Suppl. 5, pp. 18-22 (2001); Kluznik et al., J. Clin. Psychiatry, Vol. 62, Suppl. 5, pp. 14-18 (2001); Oiling et al., Biopharm. Drug Dispos., Vol. 20, pp. 19-28 (1999); Rosenbaum et al., Epilepsia, Vol. 35, pp. 656-660 (1994); Mikati et al., Epilepsia, Vol. 33, pp. 359-365 (1992); Ludden et al., Ther. Drug Monit., Vol. 13, pp. 120-125 (1991); Alvarez et al., Ann. Neurol., Vol. 9, pp. 309-310 (1981); Wilder et al., Neurology, Vol. 57, pp. 582-589 (2001); Epilepsy Foundation of America Statement on Substitution of Generic Anticonvulsant Drugs, J. Epilepsy, Vol. 1, p. 49 (1988); Nuwer et al., Neurology, Vol. 40, pp. 1647-1651 (1990); Neurology, Vol. 40, pp. 1647-1651 (1990); Cook et al., Neurology, Vol. 57, pp. 698-700 (2001); for general discussion of these issues see, Goodman and Gilman's “The Pharmacological Basis of Therapeutics”, 9^{th} Ed., Hardman and Limbird, Eds., McGraw-Hill, NY (1996), see especially Appendix 1, pp. 1697-1698 and Chapters 1 and 3).

Of course, a difference in the pharmacological effect of two drugs may be due to many factors. But it is relatively easy to determine if a given generic formulation is a pharmaceutical equivalent of a name brand product, i.e., contains the same active ingredient (same chemical entity) and in identical strength or concentration with an identical dosage form and route of administration. Moreover, this determination can be made by routine laboratory analytic procedures without administrating the drug to a patient. However, variations in the bioequivalence of otherwise pharmaceutical equivalent drugs may result in marked variations in clinical effect and these variations are much more subtle and difficult to detect except by the observation of the actual ultimate effect of the drug in real patients (see Pollak, Can. J. Cardiol., Vol. 17, No. 11, pp. 1159-1163 (2001) and Goodman and Gilman, supra). Thus this issue is of the greatest importance in connection with patient safety and economy of treatment and is of special concern to physicians as the primary factor in their choice of drug name, i.e., brand name versus generic, in writing prescription orders.

Thus, the factors that together determine the ultimate effect of a drug or treatment regimen on an organism are extremely complex and include all the pharmacokinetic and pharmacodynamic factors discussed above. In addition, clinically problematic variations in bioequivalence even between two pharmaceutically equivalent therapeutic regimens often occur and may be due to any or all of the pharmacokinetic factors described above. These factors are numerous and unpredictable and can only be determined by trial and error.

For the above reasons it is vital to be able to determine if changes in any factor that could alter bioequivalence, including but not limited to; different formulations, compositions, nature of crystal form, methods of administration or nature of excipients will produce equivalent end results in patients. Accordingly, there is a need for methods to determine and compare the actual functional clinical effect of various treatment regimens in humans so that the bioequivalence of different formulations or compositions of a given active treatment regimen can be determined with precision and reproducibility.

The final functional effect of all the drugs of interest is the production of specific and unique alterations in the gene expression profile of the exposed cell or cells of the organism. This is based, at least in part, on the discovery that alterations of various constituents of a cell, such as protein function or activity, which occur as a result of, for example, a specific drug therapy produce characteristic changes in the transcription and activity of other genes. The characteristic changes thus produced can be used to define a “signature” of the particular alterations which are correlated with the functional effects of the particular drug therapy. This is true even if there is no actual disruption in the function or activity level of proteins associated with the medication effects because of compensatory and feedback mechanisms that govern the complex process of gene regulation. Therefore, these signatures or profiles can be used to compare various aspects of drug action including those related to bioequivalence and can even be used to monitor several therapies simultaneously.

The basic concept of generating and comparing expression profiles, including gene expression profiles, to known profiles for the purpose of determining disease state, drug effectiveness and toxicity has been proposed (see, in particular; U.S. Pat. No. 5,800,992 (Fodor); U.S. Pat. No. 5,777,888 (Rine and Ashby, 1998); and U.S. Pat. No. 6,218,122 (Friend and Stoughton, 2001); and PCT Publication Nos. WO 00/39336, WO 00/39338 and WO 00/39337).

U.S. Pat. No. 6,218,122 (Friend and Stoughton, 2001) ('122), discloses methods of determining the effect of a drug therapy upon a subject by comparing a diagnostic gene expression profile from a subject undergoing a therapy with an “interpolated perturbation response profile” for that therapy. This interpolated perturbation response profile is obtained by measuring response profiles, such as gene expression profiles in analogous subject at a variety of dose levels or levels of effect of each therapy and interpolating the response profiles thus obtained. The interpolated perturbation response profile which is most similar to the diagnostic profile then indicates the level of effect or a particular therapy or drug dose level.

Thus the methods of the '122 patent allow the comparison of the gene expression profile of a subject undergoing a particular treatment regimen with an interpolated response profile that varies as a function of dose. However, it was not disclosed to use expression profiles to specifically isolate and functionally compare factors that relate only to the degree of bioequivalence between two otherwise identical, i.e., pharmaceutical equivalent, drug formulations.

PCT publication WO 00/39336 A1 (Friend Stoughton and He, International Publication Date Jul. 6, 2000) discloses methods of defining sets of co-regulated cellular constituent (called genesets) in response profiles obtained by measurement of a large number of cellular constituents in a cell or cells from a subject in response to exposure to a drug. In addition, the methods disclosed include identifying common response motifs, called consensus profiles, in the response profiles and projecting the original response profiles onto the genesets to obtain simplified, reduced-dimension response profiles which relate to drug effectiveness and toxicity.

Also disclosed in PCT publication WO 00/39336 A1 are methods for comparing a biological response to a consensus profile by use of various types of similarity metrics. These methods are used in drug finding by comparing the response profile of an unknown drug to a consensus profile. However, the disclosed methods are directed to the assessment of elements of drug action related to the pharmacodynamic properties of the drug, such as the drug activity and results of alterations in drug receptor interaction. The use of expression profile analysis to isolate and determine specifically the degree of bioequivalence between two or more pharmacologically equivalent drug formulations, in circumstances where all factors other than those which determine bioequivalence, are held constant, thus allowing measurement of the effect of variations in bioequivalence alone, is not contemplated.

Accordingly, there is a need for methods of analyzing expression profile data such as gene expression profile data or protein abundance or activity data which can isolate and compare differences in bioequivalence between supposedly similar or identical drugs or pharmaceutically equivalent drug formulations or compositions and therefore allow a simple and clinically meaningful comparison of bioequivalence to be made.

Discussion or citation of a reference herein shall not be construed as an admission that such reference is prior art to the present invention.

### SUMMARY OF THE INVENTION

The present invention provides methods for monitoring the functional result or response of two or more drug therapies upon a subject or subjects and comparing the resulting expression profiles to determine the degree of bioequivalence between the two or more drug therapies. The methods involve comparing gene or protein expression profiles, obtained by measuring gene expression products (mRNA) or protein abundances or activities in cells from a subject undergoing therapy with a first or known standard drug formulation or combination, with gene or protein expression profiles obtained by measuring mRNA or protein abundances or activities in cells of analogous subjects undergoing therapy with a second and/or unknown, but in all other respects pharmaceutical equivalent drug therapy involving the drug to be compared to the known standard.

The present invention provides methods for determining or monitoring and comparing the bioequivalence of two or more drug formulations or compositions when administered to analogous subjects comprising: (i) obtaining a first expression profile by measuring abundances of cellular constituents in a cell from a subject treated with a first drug formulation or composition; (ii) obtaining a second expression profile by measuring abundances of cellular constituents that occur in cells of an analogous subject or subjects treated with a pharmaceutically equivalent second drug formulation or composition; (iii) then comparing the thus obtained expression profiles; and (iv) determining the degree of similarity of bioequivalence between the two or more drug formulations or compositions by comparing the degree of similarity between the expression profiles, according to some objective and clinically meaningful measure.

In a further embodiment, the present invention provides methods for treating patients comprising: (i) determining the degree of bioequivalence between a first known drug formulation or composition and a second different, but pharmaceutically equivalent, drug formulation or composition; (ii) assessing, from the degree of similarity determined in (i), whether or not the second drug formulation or composition will produce a clinical result in patients that is sufficiently similar to the clinical result of the first drug formulation or composition to allow the substitution of the first drug formulation or composition with the second drug formulation or composition; and (iii) treating a patient, in need of such drug treatment, with the second drug formulation or composition if the assessment in (ii) indicates that a substitution can be made.

In yet another embodiment, the invention also provides kits for determining the bioequivalence of an unknown drug formulation or composition. These kits may comprise various combinations of components, including, but not limited to, combinations of specific oligonucleotide arrays containing in an array format containing the specific hybridization targets that are of greatest interest to the determination of the bioequivalence of a specific drug and not containing oligonucleotide targets that are not useful for that determination and databases in paper or in a computer readable medium to use in analyzing the expression profile data from these specific/unique arrays. In addition, these kits may also include various combinations of software and computer components for performing the analysis and comparison, reading and managing the data from the database and outputting the results in useful and user friendly formats.

In still another embodiment, the invention provides databases, in a paper or computer readable medium, comprising expression profile data (i.e., gene or protein expression profiles or protein activity data) for one or more drug therapies which may be used in any of the above embodiments of the invention.

In various aspects of the above embodiments, the expression profile can be determined by measuring gene expression (mRNA levels or abundances), protein abundances, protein activities, or a combination of such measurements.

In various embodiments, the methods of the invention further comprise a step of selecting only those cellular constituents that show significant response in some fraction of the expression profiles.

In a preferred aspect of the above embodiments, the expression profile is the gene expression profile obtained by simultaneous measurement of the mRNA abundances produced in response to a particular drug therapy and compared to similar measurements made in the absence of exposure to the particular drug.

Such methods are useful, e.g., in the process of drug discovery or design, for identifying drug formulations or compounds which best meet or satisfy a desired bioequivalence profile, as well as for identifying drug formulations or compounds which fall short of such a desired profile.

The methods of the present invention are also useful for analyzing modifications to known formulations or compositions that may affect bioequivalence of the resulting formulation or composition and for developing theories of why certain formulations or compositions have altered bioequivalence profiles.

The methods of the invention are also useful in treating patients. In this embodiment, the methods allow the clinician or others to choose a drug formulation or composition that, in addition to being pharmaceutically equivalent, also has very similar or identical bioequivalence to a known or standard drug formulation. Therefore, this allows the clinician to treat a patient in need of such drug treatment, with an alternative drug formulation with confidence that the alternative drug will produce a very similar or identical clinical effect on the patient, including both beneficial and toxic and/or side effects.

In addition, because the biological response to a particular drug formulation or composition will frequently vary even between different batches of that drug formulation or composition because of minute variations in the manufacturing process, the methods of the present invention are also useful during the drug manufacturing process to maintain the quality and consistency of the drug itself and to determine the degree of batch to batch variation. This would be especially valuable in the production of drugs that are derived from naturally occurring substances or semi-synthetic variations of naturally occurring substances where many difficult to control variables are involved in determining batch to batch variation.

Finally, because the biological response to particular drug formulations or composition will frequently vary between individual organisms, the methods of the present invention are also useful during treatment of an individual, e.g., in a clinical setting, to determine the best drug formulation or composition to produce a desired therapeutic effect.

In various embodiments, the methods of the invention may comprise the use of a similarity metric to compare the expression profile responses from the two or more drug formulations or compositions to be compared. The similarity metric may be the generalized cosine angle between the vectors (a) and (b), these vectors representing the plurality of cellular constituents comprising the expression profiles.

In various embodiments, the methods of the invention may further comprise the implementation of a clustering algorithm or other pattern recognition procedure to compare the two or more expression profiles and determine the similarity between them.

In various embodiments, the methods of the invention may further comprise the implementation of a clustering algorithm or other pattern recognition procedure to group the expression profiles according to similarity.

In some embodiments, the response profiles are displayed, e.g., in a false color plot, to facilitate the visual identification of similarity.

In some embodiments, the methods of the invention may make use of a “predictor set” of genes that distinguish between a known standard drug formulation or compound and a test compound with an unknown degree of variation in bioequivalent as compared to the standard drug formulation.

Finally, the methods of this invention are preferably executed on automated systems, e.g., computer system, capable of performing the above methods. Accordingly this invention also provides, in further embodiments, computer systems comprising computer-usable medium having computer readable program code embodied thereon for effecting the methods of this invention. The computer system is capable of analyzing and comparing the two or more expression profiles and determining the degree of similarity between them and producing an output that indicates the degree of bioequivalence between the test drug and the standard drug for any standard drug whose characteristic expression profile data is in the computer systems memory or can be inputted by the user.

The computer system comprises a processor, and memory coupled to said processor which encodes one or more programs. The programs encoded in memory cause the processor to perform the steps of the above methods wherein the first and second (or more) expression profiles are received by the computer system as input. In addition, the computer system memory contains the expression profile data, i.e., the database, of one or more known standard drugs in a computer readable medium.

### DESCRIPTION OF THE FIGURES

FIG. 1 illustrates an exemplary embodiment of a computer system useful for implementing the methods of the invention.

### DETAILED DESCRIPTION OF THE INVENTION

This section presents a detailed description of the invention and its applications. The description is by way of several exemplary illustrations of the general methods of the invention. The examples are non-limiting, and related variants that will be apparent to one skilled in the art are intended to be encompassed by the appended claims. In addition to these examples are descriptions of embodiments of the data gathering steps that accompany the general methods.

Expression Profile Representation

As used herein, an “expression profile” comprises measurement of a plurality of cellular constituents that indicate aspects of the biological state of a cell. Such measurements may include, e.g., RNA or protein abundances or protein activity levels.

The methods of the present invention preferably begin by measuring expression profiles. In many cases expression profiles will have already been measured in subjects for treatment with a particular drug formulation or composition. In other cases, this response data must be measured prior to the succeeding steps of this invention.

These measurements are done in analogous subjects. As used herein the term “analogous subjects” shall mean subjects similar enough to those in whom a level of therapeutic efficacy or a drug formulation effect is being determined for one skilled in the art to expect that the expression profile will be similar enough to provide useful expression profile data. In a preferred embodiment, the analogous subject is a subject of the same species and may optionally be of the same sex and/or approximate age or of the same cell type if applied to cell cultures. In certain embodiments, the analogous subjects from whom expression profiles are obtained may be the same individual (i.e., the same organism or patient) as the subject upon whom the effect of a therapy is being monitored.

As described above, the expression profiles include measurements of changes in relevant characteristics of the cellular constituents. More specifically, the ratios (or logarithms of these ratios) of native (i.e., in the absence of a drug exposure) to perturbed (i.e., in the presence of a drug exposure) gene expression levels are measured.

Expression profile data for unknown or candidate drugs are similarly obtained and must be measured if not already available. As described above, the data are obtained by measuring levels of cellular constituents in a cell of interest, i.e., a cell from a subject. The actual level of therapeutic efficacy or similarity of bioequivalence is usually unknown when this data is acquired. As above, the expression ratio is the ratio between the level in the perturbed system, and the level in the native system.

As used herein the term “drug” means any compounds of any degree of complexity that perturb a biological system, whether by known or unknown mechanisms and whether or not they are used therapeutically. Drugs thus include: typical small molecules of research or therapeutic interest; naturally-occurring factors, such as endocrine, paracrine or autocrine factors or factors interacting with cell receptors of all types; intracellular factors, such as elements of intracellular signaling pathways; factors isolated from other natural sources, such as plants and fungi, and any synthetic modifications of these isolated factors, pesticides, herbicides, insecticides and so forth.

The biological effect of a drug may be a consequence of, inter alia, drug-mediated changes in the rate of transcription or degradation of one or more species of RNA, the rate or extent of translation or post-translational processing of one or more polypeptides, the rate or extent of the degradation of one or more proteins, the inhibition or stimulation of the action or activity of one or more proteins, and so forth. In fact, most drugs exert their effects by interacting with a protein. Drugs that increase rates or stimulate activities or levels of a protein are called herein “activating drugs”, while drugs that decrease rates or inhibit activities or levels of a protein are called herein “inhibiting drugs”.

The biological effects of a drug are measured in the instant invention by observations of changes in the biological state of a cell. The cell may be of any type, e.g., prokaryotic, eukaryotic, mammalian, plant or animal. The “biological state” of a cell, as used herein, means the abundances and/or activities of a collection of cellular constituents, which are sufficient to characterize the cell for an intended purpose, such as for characterizing the effects of a drug. The measurements and/or observations made on the state of these constituents can be of their abundances (i.e., amounts or concentrations in a cell), or their activities or may be other measurements relevant to the characterization of drug action.

In various embodiments, this invention includes making such measurements and/or observations on different collections of cellular constituents. These different collections of cellular constituents are also called herein aspects of the “biological state” of the cell. As used herein, the term “cellular constituents” is not intended to refer to known subcellular organelles, such as mitochondria, lysozomes, etc.

In a preferred embodiment of the present invention, the biological state of a cell that is measured is its transcriptional state. The transcriptional state of a cell includes the identities and abundances of the constituent RNA species, especially mRNAs, in the cell under a given set of conditions. Preferably, a substantial fraction of all constituent RNA species in the cell are measured, but at least a sufficient fraction is measured to characterize the action of a drug of interest. The transcriptional state is the currently preferred aspect of the biological state measured in this invention. It can be conveniently determined by, e.g., measuring cDNA abundance s by any of several existing gene expression technologies.

Another aspect of the biological state of a cell usefully measured in the present invention is its translational state. The translational state of a cell includes the identities and abundances of the constituent protein species in the cell under a given set of conditions. As is known to those of skill in the art, the transcriptional state is often representative of the translational state. Preferably, a substantial fraction of all constituent protein species in the cell are measured, but at least, a sufficient fraction is measured to characterize the action of a drug of interest.

Other aspects of the biological state of a cell are also of use in this invention. For example, the activity state of a cell, as that term is used herein, includes the activities of the constituent protein species-(and optionally catalytically active-nucleic acid species) in the cell under a given set of conditions. As is known to those of skill in the art, the translational state is often representative of the activity state.

The present invention is also adaptable, where relevant, to “mixed” aspects of the biological state of a cell in which measurements of different aspects of the biological state of a cell are combined. For example, in one mixed aspect, the abundances of certain RNA species and of certain protein species, are combined with measurements of the activities of certain other protein species. Further, it will be appreciated from the following that this invention is also adaptable to other aspects of the biological state of the cell that are measurable.

Drug exposure will typically affect many constituents of whatever aspects of the biological state of a cell are being measured and/or observed in a particular embodiment of the invention. For example, as a result of regulatory, homeostatic, and compensatory networks and systems known to be present in cells, even an “ideal drug”, i.e., a drug that directly affects only a single constituent in a cell and without direct effects on any other constituent, will have complicated and often unpredictable indirect effects.

A drug that specifically and completely inhibits activity of a single hypothetical protein, protein Q, is considered here as an example. The drug itself may only directly change the activity of protein Q, but additional cellular constituents that are inhibited or stimulated by protein Q, or which are elevated or diminished to compensate for the loss of protein Q activity will also be affected.

Still other cellular constituents will be affected by changes in the levels or activity of the second tier constituents, and so on. Therefore, the direct effect of the drug on its target, protein Q, is hidden in the large number of indirect effects downstream from protein Q. Such downstream effects of protein Q are called herein the biological pathway originating at protein Q. Accordingly, a “non-ideal” drug that directly affects more than one primary molecular target, may have still more complicated downstream effects.

Measurement of the transcriptional state of a cell is preferred in this invention, not only because it is relatively easy to measure but also because the administration of a drug to a cell, under any specific and unique set of circumstances, will almost always result in a measurable and unique change, through direct or indirect effects, in the transcriptional state. This may be true even though the drug may act through a post-transcriptional mechanism such as inhibition of the activity of a protein or change in its rate of degradation.

A reason that drug exposure changes the transcriptional state of a cell is because the previously mentioned feed back systems, or networks, which react in a compensatory manner to infections, genetic modifications, environmental changes (including drug administration), and so forth do so primarily by altering patterns of gene expression or transcription. As a result of internal compensations, many perturbations to a biological system, although having only a muted effect on the external behavior of the system, can nevertheless profoundly influence the internal response of individual elements, e.g., gene expression, in the cell.

In some embodiments, cellular constituents are measured as continuous variables. For example, transcriptional rates are typically measured as numbers of molecules synthesized per unit time. Transcriptional rates may also be measured as percentages of a control rate. In still other embodiments, cellular constituents may be measured as categorical variables. For example, transcriptional rates may be measured as either “on” or “off”, where “on” indicates a transcriptional rate that is above or equal to a particular, user-determined threshold, and the value “off” indicates a transcriptional rate that is below that threshold.

In preferred embodiments, the response profiles analyzed by the methods of the invention are optionally screened, before the analysis, to select only those cellular constituents that have a significant response in some fraction of the profiles. In most drug treatments a large part or even a majority of these constituents will not change significantly in response to treatment, or the changes may be small and dominated by experimental error, this may be true even though the profiles may cover up to several hundred or thousand cellular constituents. In most embodiments it will be useful to delete these constituents from all profiles in the analysis methods of the invention.

In some embodiments, only cellular constituents that have a response greater than or equal to two standard errors in more than N profiles are selected for subsequent analysis, where N may be one or more and is preferably selected by the user. Preferably, N will tend to be larger for larger sets of response profiles. For example, in one preferred embodiment N may be approximately equal to the square root of the number of response profiles being analyzed.

In some embodiments the cellular constituents and/or the expression profiles may be visually displayed, e.g., in a false color plot indicating increases and/or decreases in activity levels and/or abundances of each cellular constituent. For example, in some embodiments wherein the cellular constituents comprise genetic transcripts, such a visual display would preferably comprise a false color plot of up-regulation and down-regulation of individual transcripts.

Within eukaryotic cells, there are hundreds to thousands of signaling pathways that are interconnected. For this reason, any perturbations in the function of proteins within a cell will have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug under specific circumstances, will result in characteristic and unique compensatory changes in the transcription of enough other genes that these changes in transcripts can be used to define a “signature” of particular transcript alterations which are related to the specific disruption of function caused by the unique characteristics of the particular drug exposure, even at a stage where changes in protein activity are undetectable.

In fact, such compensatory changes can be monitored long before it is possible to detect changes by monitoring protein function. The resultant up regulation and down regulation of genes within a cell when the biological state of the cell is disrupted or partially disrupted represent compensatory changes that the cell undertakes in order to maintain homeostasis. As these compensatory changes in transcription occur before the cell exhibits any discernable physiological change, these expression profiles are very sensitive indications of the cell's biological state. This sensitivity has a significant value when it comes to detecting and comparing the specific and unique effects of a drug exposure and allows the isolation of subtle effects of alterations in bioequivalence from the effects of all other alterations in the nature of the drug exposure, including, but not limited to, variations in any pharmacodynamic properties of the drugs to be compared.

Expression profiles for monitoring efficacy of a therapy can be generated and measured by measuring cellular constituents in analogous subject or subjects undergoing an identical therapy. Passive procedures for obtaining the required gene expression profiles and protein activity data are therefore employed in such systems. These passive procedures for obtaining gene expression profiles and protein activity data include, but are not limited to, taking tissue or blood samples from individuals before and after undergoing regimens of drug treatment for investigational, therapeutic or other purposes at fixed or varying drug dosages.

Although, much of the description of this invention is directed to measurement and modeling of gene expression data, this invention is equally applicable to measurements of other aspects of the biological state of a cell, such a protein abundances or activities. Methods for direct measurement of protein activity are well known to those of skill in the art. Such methods include, e.g., methods which depend on having an antibody ligand for the protein, such as Western blotting (see, e.g., Burnette, Anal. Biochem., Vol. 112, pp. 195-203 (1981)).

Such methods also include enzymatic activity assays, which are available for most well-studied protein drug targets, including, but not limited to, HMG CoA reductase (Thorsness et al., Mol. Cell. Biol., Vol. 9, pp. 5702-5712 (1989)), and calcineurin (Cyert et al., Mol. Cell.

Biol., Vol. 12, pp. 3460-3469 (1992)). An example of turning off a specific gene function by turning off the controllable promoter, and correlating this with protein depletion via Western blotting is given in Deshaies et al., Nature, Vol. 332, pp. 800-805 (1988).

Analytical Methods of the Invention

Method One: The Vector Comparison Method

Analytical embodiments of the methods and systems of the present invention include, first, embodiments for representing measured biological expression profiles, especially measured gene or protein expression profiles of a biological sample to an exposure to two or more pharmaceutically equivalent drug formulations in terms of “vectors”of multiple cellular constituents.

An additional aspect of the analytical embodiments of this invention comprises embodiments for determining the similarity (by various measures) of the measured expression profile “vectors” and determining an objective measure of this similarity and therefore the bioequivalence of the thus compared drug formulations or compositions, since all other relevant factors are held constant by the methods of administration of the two or pharmaceutically equivalent drug formulations or compounds to be compared.

This invention also includes kits for the determination of bioequivalence comprising specific nucleotide arrays and databases in computer readable medium to be used in combination with the computer systems.

The embodiments of the analytical methods of the invention are preferably done using automated systems, e.g., computer systems, which perform one or more of the methods of the various embodiments of the invention automatically given user input comprising, e.g., expression profile data from one or more biological samples.

Representation of Biological Responses as Vectors

The response of a biological sample (e.g., a cell, cell culture or an organism) to the application of a drug can be measured by observing changes in the biological state of the sample. For example, response data are obtained by a method involving treating one or more biological samples (e.g., cells or organisms) with two or more different but pharmaceutically equivalent drug formulations to produce characteristic drug response expression profiles involving a well-defined set of cellular constituents. The term “biological sample” as used herein, shall mean a cell, a group of cells such as a cell culture or an intact organism. The application of the two or more drug formulations to the biological samples is conducted in such a manner that the only differences between the two or more drug treatments is due to variations in bioequivalence between the drug formulations, i.e., the drug formulations and methods of administration constitute “pharmaceutically equivalent” treatments. In practice, to reduce spurious results, in some embodiments only those mRNA transcripts with responses larger than a certain factor are included. In a preferred embodiment this is a factor of two.

Such expression profile data comprise a collection of measured changes of a plurality k of cellular constituents. Such expression profiles can be described for quantitative analysis in terms of the vector v. In particular, the expression profile of a biological sample to the perturbation n is defined herein as the vector v⁽ⁿ⁾.
v⁽ⁿ⁾=[vᵢ⁽ⁿ⁾, . . . , vᵢ⁽ⁿ⁾, . . . vₖ⁽ⁿ⁾] (1)
where vᵢ⁽ⁿ⁾ is the amplitude of the response of cellular constituent i under the perturbation, i.e., drug exposure, n. In some embodiments, vᵢ⁽ⁿ⁾ may be simply the difference between the abundances and/or activity levels of cellular constituent i before and after the perturbation n is applied to the biological sample, or the difference in abundances and/or activity levels of cellular constituent i between a biological sample that is subject to the perturbation n and a sample that is not subject to the perturbation n.

In other embodiments, vᵢ⁽ⁿ⁾ is the ratio (or the logarithm of the ratio) of the abundances and/or activity levels of cellular constituent i before and after the perturbation n is applied to the biological sample, or the ratio (or the logarithm of the ratio) of abundance and/or activity level of cellular constituent i in a sample subject to the perturbation n to a sample that is not subject to the perturbation n.

In preferred embodiments, vᵢ⁽ⁿ⁾ is set equal to zero for all cellular constituents i whose response is below a threshold amplitude or confidence level which may be determined, e.g., from knowledge of the measurement error behavior. For example, in some embodiments, only cellular constituents that have a response greater than or equal to two standard errors in more than n profiles may be selected for subsequent analysis, where the number of profiles n is preferably selected by a user of the invention.

For those cellular constituents whose responses are above a given threshold amplitude, vᵢ⁽ⁿ⁾ may simply be made equal to the measured value. For example, in embodiments wherein the perturbation n comprises exposure to a specific drug, then the response vᵢ⁽ⁿ⁾ may simply be made equal to the expression and/or activity of the Ah cellular constituent whose activity is altered above the threshold amplitude by the administration of the drug.

In an alternative embodiment, the response profile data may be categorical. For example, in a binary approximation the response amplitude vᵢ⁽ⁿ⁾ is set equal to unity if cellular constituent i has a significant response to perturbation n, and is set equal to zero if there is no significant response. Alternatively, in a trinary approximation the response amplitude is set equal to +1 if cellular constituent i has a significant increase in expression and/or activity to perturbation n, is set equal to zero if there is no significant response, and is set equal to −1 if there is a significant decrease in expression and/or activity. Such embodiments are particularly preferred if it is known or suspected that the individual components of the vector response to which the vector v⁽ⁿ⁾ is to be compared do not have the same relative amplitudes as in v⁽ⁿ⁾ but do involve the same cellular constituents.

In yet other embodiments, it may be desirable to use “Mutual Information”, as described and enabled, e.g., by Brunel, Neural Computation, Vol. 10, No. 7, pp. 1731-1757 (1998)).

In the above embodiments, it may be preferred to normalize the response expression profile by scaling all elements of the vector v⁽ⁿ⁾ (i.e., vᵢ⁽ⁿ⁾ for all I) by the same constant so that the vector length |v⁽ⁿ⁾| is unity. Generally, the vector length may be defined by

$${\vert {v}^{( n )} \vert}^{2} = {\sum }_{i} {( {V}_{i}^{( n )} )}^{2} \qquad ( 2 )$$

Methods to Compare Expression Profile Vectors to Determine Bioequivalence
Use of the Generalized Cosine Angle as a Similarity Metric

Once the characteristic expression profile for a standard drug formulation has been identified, similarities and differences between two or more expression profiles, representing the effects of the administration of different but pharmaceutically equivalent drug formulations or compositions may be readily evaluated and compared to determine similarity and therefore the degree of bioequivalence between the two or more drug formulations. Here we use P^{(a)} to indicate the vector representation of the expression profile of a known standard drug formulation, i.e., drug “formulation a” and we use P^{(b} to represent the vector representation of the expression profile of an unknown and/or candidate or alternative drug formulation or composition, i.e., drug “formulation b”.

Those of skill in the art are aware of many mathematical methods by which two or more such vectors can be compared and a determination of similarity made. In preferred embodiments, the two or more expression profile vectors are compared by an objective, quantitative similarity metric S. In one particularly preferred embodiment, the similarity metric S is the generalized cosine angle between the two (or more) expression profile vectors being compared, in this example between P^{(a)} and P^{(b)}.

The generalized cosine angle is a metric well known to those skilled in the art, and may be defined by the equation

$${S}_{a , b} = S ( {P}^{( a )} , {P}^{( b )} ) = \frac{{P}^{( a )} \cdot {P}^{( b )}}{\vert {P}^{( a )} \vert \vert {P}^{( b )} \vert} \qquad ( 3 )$$

wherein the dot product, P^{(a)}·P^{(b)}, is defined by

$${P}^{( a )} \cdot {P}^{( b )} = {\sum }_{q} ( {P}_{q}^{( a )} \times {P}_{q}^{( b )} \qquad ( 4 )$$

and
|P^{(a)}=(P^{(a)}·P^{(a)})^{1/2}, and |P^{(b)}|=(P^{(b)}·P^{(b)})^{1/2}.

In such embodiments, expression profile vector P^{(a)} is most similar to expression profile vector P^{(b)}, if S_{a,b} is a maximum. In this embodiment S_{a,b}, constitutes a “correlation coefficient” and may have any value between −1 to +1. A value of S_{a,b}=+1 indicates that the two profiles are essentially identical; the same cellular constituent effected in P^{(a)} are proportionally effected in P^{(b)}, although the magnitude (i.e., strength) of the two responses may be different. A value of S_{a,b}=−1 indicates that the two profiles are essentially opposites. Thus, although the same cellular constituent sets in P^{(a)} are proportionally effected in P^{(b)}, those sets which increase (e.g., are up-regulated) in P^{(a)} decrease (e.g., are down regulated) in P^{(b)} and vice-versa. Such profiles are said to be “anti-correlated”. Finally, a value of S_{a,b}=0 indicates maximum dissimilarity between the two responses; those cellular constituent sets effected in P^{(a)} are not effected in P^{(b)} and vice-versa.

Determination of Bioequivalence from Vector Similarity Comparisons

In general, the expression profiles produced by the techniques of this invention constitute arrays of numbers which indicate the abundance or activity, or change in abundance or activity or the ratio (or the logarithm of the ratio) of the change in abundance or activity of various cellular constituents produced in response to contact with a drug or some other disturbing influence. In one preferred embodiment the ratio of the pre and post administration expression rates (or abundances over a fixed period of time) of a plurality of mRNA species whose expression is altered by the administration of a specific drug formulation, constitutes one type of expression profile. The methods described above convert these expression profiles into vector representations and then compare these vectors to produce a measure of similarity called a correlation coefficient.

The goal of this comparison would include, but not be limited to, determining if two or more different, even though perhaps very similar formulations of a given drug, i.e., pharmaceutical equivalents, are similar enough in clinical effect in patients to allow substitution of a new or modified formulation of a given drug, call it formulation B, for the standard formulation of the drug, call this formulation A, without significant changes in the clinical effects of the resulting treatment, including beneficial or desired effects, side effects and toxic or otherwise adverse or undesirable effects.

This comparison would be done in the following fashion. First, the formulation to be compared to formulation A would be administered to analogous subjects under circumstances which would allow pharmaceutical equivalence to be maintained. This would require that the two formulations, A and B contain the same active ingredients (chemically identical active drug substance or substances) and be identical in strength or concentration, dosage form, and route of administration and that the subjects be “analogous”.

Under these circumstances the pharmaceutical effects of the two formulations will be identical if and only if these two pharmaceutically equivalent drug formulations are also “bioequivalent”, meaning that the rates and extents of bioavailability of the active ingredient(s) in the two drug formulations are not significantly different under these otherwise identical or very similar test conditions. The alterations in the expression profile in the subjects receiving formulation B would then be determined in precisely the same manner as it was for formulation A described above. The result of this determination would be the production of an expression profile for formulation B containing all or most of the elements as the expression profile for formulation A. In one embodiment these elements would consist of the abundances, the ratio of the abundances or the logarithm of the ratio of the altered mRNA species. In other embodiments these elements could comprise protein abundances or ratios of abundances or protein activity or ratios of protein activity.

The expression profile for formulation A, already determined, could be stored in the memory of one of the embodiments of the computer systems disclosed or could be inputted by the user at the time of the comparison process. The expression profile for formulation B would then be inputted into the computer system by any method and the software would then perform the actions required to determine the degree of bioequivalence of the two formulations.

These actions would comprise; causing execution of expression profile analysis software which performs the steps of causing the computer's processor to execute steps of (a) receiving one or more expression profiles, which may come from user input or from the internal database; (b) calculating the equivalent vector representations; (c) comparing these vectors to determine their degree of similarity, i.e., the correlation coefficient; (d) calculating bioequivalence based on this degree of similarity; and (e) outputting this calculated bioequivalence in a user friendly and clinically relevant format. Of course, the calculation described above could also be done by hand and without the use of any computer or automated system.

The step of comparing the vectors to determine their degree of similarity could be accomplished by any of the mathematical methods disclosed herein. In a preferred embodiment the computer would calculate the generalized cosine angle between the two vector representations of the multi-element expression profile vectors and thereby provide a correlation coefficient with a value of from −1 to +1. A correlation coefficient of +1 would indicate that the two vectors were identical while a coefficient of 0 would indicate no relationship between the two vectors. A coefficient of −1 would indicate a perfect negative association and therefore negative coefficients are, in this example, not relevant. The sensitivity, with regard to the underlying biology, of this technique would be extremely high so that even small deviations from a correlation coefficient of +1 would indicate significant and clinically relevant variations in bioequivalence between formulations A and B.

One of skill in the art can devise many ways to determine, for a given biology, what effect a given variation in the value of the correlation coefficient has on the clinical effect of the drug formulation under comparison. For example, minor variations in the formulation of a known drug could be made and tested for clinical effect in patients or in an appropriate animal model. Ideally these altered formulations would be pharmaceutical equivalents of each other but differ slightly in the subtle factors that determine bioequivalence. The clinical variations observed could be correlated with the measured correlation coefficient for the two formulations to produce an empirical determination of the clinical significance of a correlation coefficient less than one.

A similar determination could be made by measuring correlation coefficients for drug formulations which are known to be clinically none-equivalent despite being pharmaceutical equivalents. Examples of such drug formulations would be drug Clozaril and the supposedly bioequivalent generic version of clozapine, other well known examples include a variety of anticonvulsant medications (see Ronald et al., Neurology, Vol. 57, pp. 571-573 (2001); Keith, Int. J. Fertil. Womens. Med., Vol. 46, No. 6, pp. 286-295 (2001); Balter et al., Clin.

Ther., Vol. 23, No. 10, pp. 1720-1731 (2001); Lam, J. Clin. Psychiatry, Vol. 62, Suppl. 5, pp. 18-22 (2001)).

Other means to correlate the value of the correlation coefficient determined by the methods of this invention with clinically relevant variations in therapeutic efficacy or toxic or side effects will be readily apparent to those of skill in the art.

For the purposes of this invention a correlation coefficient of less than a given amount, depending on the individual biology, would indicate a clinically relevant lack of bioequivalence between the two formulation and this would dictate that formulation B could not be substituted for formulation A without significant and possibly dangerous changes in the efficacy of the resulting treatment. Therefore, two pharmaceutically equivalent drug formulations would be considered to be sufficiently bioequivalent to allow substitution or to demonstrate satisfactory consistency in, for example, batch to batch drug formulation determinations, if the correlation coefficient is, in one embodiment greater than 0.90, in a preferred embodiment the correlation coefficient would be greater that 0.95, in a more preferred embodiment the correlation coefficient would be greater than then 0.98, and in a most preferred embodiment the correlation coefficient would be greater than 0.99.

Method Two: The Predictor Gene Set Method

A second method of determining the degree of bioequivalence involves first establishing a “predictor set” of genes that distinguish between the known standard drug formulation or compound and a test compound with a known degree of variation in bioequivalence as compared to the standard drug formulation. This test compound may be an inactive compound, such as a placebo or in a preferred embodiment this drug formulation would be a drug that is very similar to the standard compound, preferably a pharmaceutical equivalent, but a compound that is known not to be completely bioequivalent. Examples, as discussed above, would include but not be limited to, a drug such as Clozaril and the supposedly bioequivalent generic version of clozapine, other examples, well known to clinicians, would include a variety of anticonvulsant medications (see, e.g., Ronald et al., Neurology, Vol. 57, pp. 571-573 (2001); Keith, Int. J. Fertil. Womens. Med., Vol. 46, No. 6, pp. 286-295 (2001); Balter et al., Clin. Ther., Vol. 23, No. 10, pp. 1720-1731 (2001); Lam, J. Clin. Psychiatry, Vol. 62, Suppl. 5, pp. 18-22 (2001)).

This predictor set of genes can then be used to compare the known standard drug formulation with an unknown drug formulation. This method will be able to determine whether or not the unknown drug is more or less similar in bioequivalence in comparison to the standard drug formulation as the original test compound was. In a preferred embodiment the test compound used to develop the predictor set of genes would be a compound that is a pharmaceutical equivalent of the standard formulation but a compound whose bioequivalence is known, on clinical or other grounds, to differ from the standard drug formulation to a degree that is detectable, quantifiable and clinically relevant.

To perform the comparison according to this method gene expression profiles from multiple biological samples, which may be human patients or test animals or cell cultures, some treated with the standard drug formulation and some treated with the non-bioequivalent formulation (a total of “n” samples) would be compiled. The resulting data would be filtered to eliminate those genes that are not significantly different between biological samples treated with active compound and those treated with inactive (or non-bioequivalent) compound, for example, by use of statistical tests such as non-parametic ANOVA. This filtered list of genes would then be ordered according to their correlation with the treatment status, i.e., whether the sample had been treated with or exposed to the standard drug formulation (Active compound) or to the placebo or drug formulation known not to be bioequivalent to the standard formulation (Inactive compound), then:
Assign a numeric value to each treatment type: I=Active (standard) compound; 0=Inactive (non-bioequivalent) compound.
For each gene, determine Pearson correlation (see below) between expression value and treatment status using all of the samples.

The Pearson product moment correlation coefficient, r, is a dimensionless index that ranges from −1.0 to 1.0 inclusive and reflects the extent of a linear relationship between two data sets.

The r value of the regression line is:

$$r = \frac{Σ X Y - \frac{Σ X Σ Y}{N}}{\sqrt{( Σ {X}^{2} - \frac{{( Σ X )}^{2}}{N} ) ( Σ {Y}^{2} - \frac{{( ΣY )}^{2}}{N} )}}$$

The absolute value of the correlation coefficient is used so as to give equal weight to both positive and negative correlations with treatment status. Then the genes are ordered by absolute correlation coefficient, from highest to lowest.

Then a “leave-out-out” strategy is applied to determine the optimum number of genes to use as the final predictor set (see, van't Veer, et al., “Gene Expression Profiling Predicts Clinical Outcome of Breast Cancer”, Nature, Vol. 415, pp. 530-536 (2002)). This entails defining a model set of genes and testing the model by leaving one sample out at a time to determine how well the model fits, this is done as follows:
Starting with the top 5 genes (those with the highest correlation to treatment status), take one sample out of the analysis, and calculate the mean gene expression profile for each treatment group (Active and Inactive) from the remaining samples (n-1).
Predict the outcome for the left-out sample by determining the Pearson correlation of the expression profile of the left out sample with the mean Active and Inactive treatment profiles calculated using the n−1 samples.
If the correlation coefficient is higher when compared with the Active treatment profile than when compared to the Inactive profile, the sample would be classified as Active and assigned a value of 1.
If the correlation coefficient is higher when compared with the Inactive treatment profile than when compared to the Active profile, the sample would be classified as Inactive and assigned a value of 0.
Repeat this analysis using the remaining samples until all n samples had been left out once.
Determine the number of cases of correct and incorrect predictions by calculating the number of false negatives (Active mis-classified as Inactive) and false positives (Inactive mis-classified as Active).
Repeat the “leave-one-out” process after adding additional predictor genes one at a time, from the top of the list, until all of the genes have been used.
The number of genes that results in the lowest error rates (both false positives and false negatives) defines the model to use as the gene predictor set.

Using this optimized number of predictor genes, the next step is to calculate the appropriate threshold value to use for an accurate determination of the bioequivalence of drug formulations:
Using all of the samples treated with the Active (standard drug formulation) compound, calculate the average expression value for each of the optimized predictor genes. This is defined as the “mean Active expression profile”.
For each sample, calculate the Pearson correlation between expression values for the predictor genes to the mean Active expression profile.
Rank the samples by correlation coefficient, from highest to lowest, such that those samples at the top of the list are more closely correlated with the Active expression profile and those samples at the bottom of the list would be least correlated with the Active expression profile (and thus labeled Inactive).
Calculate error rates of prediction (false negatives and false positives) using all potential threshold values of correlation coefficient.
The threshold at “optimal accuracy” would be defined as that correlation coefficient cutoff value that resulted in the fewest number of both false negatives and false positives. However, in some embodiments it may be desirable to optimize the threshold for sensitivity, such as reducing the number of false negatives (at the expense of increased false positives).

To determine if a new or unknown drug formulation B is bioequivalent to a standard drug formulation A, an analogous biological sample would be contacted or treated with the unknown formulation under conditions identical to those employed for the establishment of the mean Active expression profile. The formulation to be compared, formulation B, to the standard drug formulation A would be administered to analogous subjects (or biological samples) under circumstances which would allow pharmaceutical equivalence to be maintained. This would require that the two formulations, A and B, contain the same active ingredients (chemically identical active drug substance or substances) and be identical in strength or concentration, dosage form and route of administration and that the subjects be “analogous”. The gene expression profile of the predictor genes as determined above would then be compared to the mean Active expression profile for the standard drug formulation and the Pearson correlation determined:
If the correlation coefficient for a given test sample was greater than or equal to the predetermined threshold value (see above), then that sample would be classified as “Active”; if the correlation coefficient was less than the threshold the sample would be classified as “Inactive”.

A unknown drug formulation B would be classified as “bioequivalent” if a significant number, e.g., a majority, of the test samples were classified as “Active”.

This classification would constitute a determination that the unknown drug formulation was as similar or more similar in bioequivalence to the standard drug formulation as was the original test compound used to define the predictor set of genes. Conversely, the finding of a correlation coefficient less than the predetermined threshold value would constitute a determination that the unknown drug formulation showed a greater difference in bioequivalence than the original test compound.

Therefore, if the original unknown drug formulation used to develop the gene predictor set, was known, on clinical grounds to be a fully bioequivalent substitution for the standard drug formulation and the drug under test is classified as active, that is if the correlation coefficient as determined above is greater than or equal to the predetermined threshold value then this test drug would also be a fully bioequivalent substitution for the standard drug formulation. If the correlation coefficient as determined above is less than the predetermined value then the drug under test would be expected to be less than a fully bioequivalent substitution for the standard drug formulation and may be markedly different in its clinical effect in patients.

The results of this type of analysis would become more difficult to interpret as the unknown drug formulation used to develop the gene predictor set differs more and more from the standard drug formulation. Therefore, in a preferred embodiment the test compound used to develop the predictor set of genes would be a compound that is a pharmaceutical equivalent of the standard formulation and a compound whose bioequivalence differs a known and clinically relevant degree from that of the standard drug formulation.

As described for the previous method in a preferred embodiment the above steps would be performed by the computer system described herein. The expression profile for the predictor genes, already determined, could be stored in the memory of one of the embodiments of the computer systems disclosed or could be inputted by the user at the time of the comparison process. The expression profile(s) for the unknown drug formulation B would then be inputted into the computer system by any method and the software would then perform the actions required to determine the degree of bioequivalence of the two formulations.

Measurement Methods and Arrays

The experimental methods of this invention depend on measurements of cellular constituents. The cellular constituents measured can be from any aspect of the biological state of a cell. They can be from the transcriptional state, in which RNA abundances are measured, the translation state, in which protein abundances are measured, the activity state, in which protein activities are measured. The cellular characteristics can also be from mixed aspects, for example, in which the activities of one or more proteins are measured along with the RNA abundances (gene expressions) of other cellular constituents. This section describes exemplary methods for measuring the cellular constituents in drug or pathway responses. This invention is adaptable to other methods of such measurement.

Preferably, in this invention the transcriptional state of the other cellular constituents is measured. The transcriptional state can be measured by techniques of hybridization to arrays of nucleic acid or nucleic acid mimic probes, described in the next subsection, or by other gene expression technologies, described in the subsequent subsection. However measured, the result is data including values representing mRNA abundance and/or ratios, which usually reflect DNA expression ratios (in the absence of differences in RNA degradation rates).

In various alternative embodiments of the present invention, aspects of the biological state other than the transcriptional state, such as the translational state, the activity state or mixed aspects can be measured.

Measurement of Transcriptional State

Preferably, measurement of the transcriptional state is made by hybridization of nucleic acids to oligonucleotide arrays, which are described in this subsection. Certain other methods of transcriptional state measurement are described later in this subsection. In all embodiments, measurements of the cellular constituents should be made in a manner that is relatively independent of when the measurements are made.

Transcript Arrays Generally

In a preferred embodiment, the present invention makes use of “oligonucleotide arrays” (also called herein “microarrays”). Microarrays can be employed for analyzing the transcriptional state in a cell, and especially for measuring the transcriptional states of cancer cells.

In one embodiment, transcript arrays are produced by hybridizing detectably labeled polynucleotides representing the mRNA transcripts present in a cell (e.g., fluorescently-labeled cDNA synthesized from total cell mRNA or labeled cRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics. The arrays are reproducible, allowing multiple copies of a given array to be produced and easily compared with each other. Preferably the microarrays are small, usually smaller than 5 square cm and they are made from materials that are stable under binding (e.g., nucleic acid hybridization) conditions.

A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. In a specific embodiment, positionally addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA or cRNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

Preparation of Microarrays

Microarrays are known in the art and consist of a surface to which probes that correspond in sequence to gene products (e.g., cDNAs, mRNAs, cRNAs, polypeptides and fragments thereof, can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA or cRNA can specifically hybridize. The nucleic acid or analogue of the binding site can be, e.g., a synthetic oligomer, a full-length cDNA, a less-than full-length cDNA or a gene fragment.

Although in a preferred embodiment the microarray contains binding sites for products of all or almost all genes in the target organism's genome, such comprehensiveness is not necessarily required. The microarray may have binding sites for only a fraction of the genes in the target organism. However, in general, the microarray will have binding sites corresponding to at least about 50% of the genes in the genome, often at least about 75%, more often at least about 85%, even more often more than about 90%, and most often at least about 99%. Preferably, the microarray has binding sites for genes relevant to testing and confirming a biological network model of interest.

A “gene” is identified as an open reading frame (ORF) of preferably at least 40, 80 or 99 amino acids from which a mRNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6,275 ORFs longer than 99 amino acids. Analysis of these ORFs indicates that there are 5,885 ORFs that are likely to specify protein products (see Goffeau et al., “Life With 6000 Genes”, Science, Vol. 274, pp. 546-567 (1996), which is incorporated by reference in its entirety for all purposes). In contrast, the human genome is estimated to contain approximately 25,000-35,000 genes.

Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences or the sequences may be synthesized de novo on the surface of the chip, for example by use of photolithography techniques, e.g., Affymetrix uses such a different technology to synthesize their oligos directly on the chip). PCR primers are chosen, based on the known sequence of the genes or cDNA, that result in amplification of unique fragments (i.e., fragments that do not share more than 10 bases of contiguous identical sequence with any other fragment on the microarray).

Computer programs are useful in the design of primers with the required specificity and optimal amplification properties (see, e.g., Oligo pl version 5.0, National Biosciences). In the case of binding sites corresponding to very long genes, it will sometimes be desirable to amplify segments near the 3′-end of the gene so that when oligo-dT primed cDNA-probes are hybridized to the microarray; less-than-full length probes will bind efficiently. Typically each gene fragment on the microarray will be between about 20 bp and about 2,000 bp, more typically between about 100 bp and about 1,000 bp, and usually between about 300 bp and about 800 bp in length. PCR methods are well known and are described, for example, in Innis et al., Eds., “PCR Protocols: A Guide to Methods and Applications”, Academic Press Inc., San Diego, Calif. (1990), which is incorporated by reference in its entirety for all purposes. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (see Froehler et al., Nuc. Acid Res., Vol. 14, pp. 5399-5407 (1986); McBride et al., Tetra. Lett., Vol. 24, pp. 245-248 (1983)). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., “PNA Hybridizes to Complementary Oligonucleotides Obeying the Watson-Crick Hydrogen-Bonding Rules”, Nature, Vol. 365, pp. 566-568 (1993); see also U.S. Pat. No. 5,539,083).

In an alternative embodiment, the binding (hybridization) sites are made from plasmid or phage clones of genes, cDNAs (e.g., expressed sequence tags), or inserts therefrom (see Nguyen et al., “Differential Gene Expression in The Murine Thymus Assayed by Quantitative Hybridization of Arrayed cDNA Clones”, Genomics, Vol. 29, pp. 207-209 (1995)). In yet another embodiment, the polynucleotide of the binding sites is RNA.

Attaching nucleic acids to the solid surface The nucleic acid or analogue are attached to a solid support, which may be made from glass, plastic (e.g., polypropylene, nylon), polyacrylamide, nitrocellulose or other materials.

A preferred method for attaching the nucleic acids to a surface is by printing on glass plates, as is described generally by Schena et al., “Quantitative Monitoring of Gene Expression Patterns With a Complementary DNA Microarray”, Science, Vol. 270, pp. 467470 (1995).

This method is especially useful for preparing microarrays of cDNA (see, also, DeRisi et al., “Use of a cDNA Microarray to Analyze Gene Expression Patterns in Human Cancer”, Nature Genetics, Vol. 14, pp. 457-460 (1996); Shalon et al., “A DNA Microarray System For Analyzing Complex DNA Samples Using Two-Color Fluorescent Probe Hybridization”, Genome Res., Vol. 6, pp. 639-645 (1996); and Schena et al., “Parallel Human Genome Analysis; Microarray-Based Expression of 1000 Genes”, Proc. Natl. Acad. Sci. USA, Vol. 93, pp. 10539-11286 (1995)). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., “Light-Directed Spatially Addressable Parallel Chemical Synthesis”, Science, Vol. 251, pp. 767-773 (1991); Pease et al., “Light-Directed Oligonucleotide Arrays For Rapid DNA Sequence Analysis”. Proc. Natl. Acad. Sci. USA, Vol. 91, pp. 5022-5026 (1994); Lockhart et al., “Expression Monitoring by Hybridization to High-Density Oligonucleotide Arrays”, Nature Biotech., Vol. 14, p. 1675 (1996); U.S. Pat. Nos. 5,578,832; 5,556,752; and 5,510,270, each of which is incorporated by reference in its entirety for all purposes) or other methods for rapid synthesis and deposition of defined oligonucleotides (see Blanchard et al., “High-Density Oligonucleotide Arrays”, Biosensors & Bioelectronics, Vol. 11, pp. 687-690 (1996)). When these methods are used, oligonucleotides (e.g., 25-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (see Maskos et al., Nuc. Acids Res., Vol. 20, pp. 1679-1684 (1992)), may also be used. In principal, any type of array, for example, dot blots on a nylon hybridization membrane (see Sambrook et al., “Molecular Cloning—A Laboratory Manual (2nd Ed.)”, Vols. 1-3, Cold Spring Harbor Laboratory, Cold Spring Harbor, N.Y. (1989), which is incorporated in its entirety for all purposes), could be used, although, as will be recognized by those of skill in the art, very small arrays will be preferred because hybridization volumes will be smaller.

Generating Labeled Probes

Methods for preparing total and poly(A)⁺ RNA are well known and are described generally in Sambrook et al., supra. In one embodiment, RNA is extracted from cells of the various types of interest in this invention using guanidinium thiocyanate lysis followed by CsCl centrifugation (see Chirgwin et al., Biochemistry, Vol. 18, pp. 5294-5299 (1979)). Poly(A)⁺ RNA is selected by selection with oligo-dT cellulose (see Sambrook et al., supra). Cells of interest include wild-type cells, drug-exposed wild-type cells, cells with modified/perturbed cellular constituent(s) and drug-exposed cells with modified/perturbed cellular constituent(s).

Labeled cDNA is prepared from mRNA or alternatively directly from RNA by oligo dT-primed or random-primed reverse transcription, both of which are well known in the art (see, e.g., Klug et al., Methods Enzymol., Vol. 152, pp. 316-325 (1987)). Reverse transcription may be carried out in the presence of a dNTP conjugated to a detectable label, most preferably a fluorescently-labeled dNTP. Alternatively, isolated mRNA can be converted to labeled antisense RNA synthesized by in vitro transcription of double-stranded cDNA in the presence of labeled dNTPs (see Lockhart et al., “Expression Monitoring by Hybridization to High-Density Oligonucleotide Arrays”, Nature Biotech., Vol. 14, p. 1675 (1996), which is incorporated by reference in its entirety for all purposes). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, “Nonisotopic DNA Probe Techniques”, Academic Press, San Diego, Calif. (1992)). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., “High Density cDNA Filter Analysis: A Novel Approach For Large-Scale, Quantitative Analysis of Gene Expression”, Gene, Vol. 156, p. 207 (1995); Pietu et al., “Novel Gene Transcripts Preferentially Expressed in Human Muscles Revealed by Quantitative Hybridization of a High Density cDNA Array”, Genome Res., Vol. 6, p. 492 (1996)). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, DATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript. TM: II, LTI Inc.) at 42° C. for 60 minutes.

Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when; if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., supra).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-Interscience, NY (1987), which is incorporated in its entirety for all purposes. When the cDNA microarrays of Schena et al. are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1×SSC plus 0.2% SDS) (see Shena et al., Proc. Natl. Acad. Sci. USA, Vol. 93, p. 10614 (1996)). Useful hybridization conditions are also provided in, e.g., Tijessen, “Hybridization With Nucleic Acid Probes”, Elsevier Science Publishers B.V. (1993) and Kricka, “Nonisotopic DNA Probe Techniques”, Academic Press, San Diego, Calif. (1992).

Signal Detection and Data Analysis

When fluorescently-labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows specimen illumination at wavelengths specific to the fluorophores used and emissions from the fluorophore can be analyzed. In a preferred embodiment, the arrays are scanned with a laser fluorescent scanner with a computer controlled X-Y stage and a microscope objective. Sequential excitation of the fluorophore is achieved with a multi-line, mixed gas laser and the emitted light is split by wavelength and detected with a photomultiplier tube. Fluorescence laser scanning devices are described in Schena et al., Genome Res., Vol. 6, pp. 639-645 (1996) and in other references cited herein. Alternatively, the fiber-optic bundle described by Ferguson et al., Nature Biotech., Vol. 14, pp. 1681-1684 (1996), may be used to monitor mRNA abundance levels at a large number of sites simultaneously.

Signals are recorded and, in a preferred embodiment, analyzed by computer, e.g., using a 12-bit analog to digital board. In one embodiment the scanned image is despeckled using a graphics program (e.g., Hijaak Graphics Suite) and then analyzed using an image gridding program that creates a spreadsheet of the average hybridization at each wavelength at each site.

The Agilent Technologies GENEARRAY™ scanner is a bench-top, 488 nM argon-ion laser-based analysis instrument. The laser can be focused to a spot size of less than 4 microns. This precision allows for the scanning of probe arrays with probe cells as small as 20 microns. The laser beam focuses onto the probe array, exciting the fluorescent-labeled nucleotides. It then and then scans using the selected filter for the dye used in the assay. Scanning in the orthogonal coordinate is achieved by moving the probe array. The laser radiation is absorbed by the dye molecules incorporated into the hybridized sample and causes them to emit fluorescence radiation. This fluorescent light is collimated by a lens and passes through a filter for wavelength selection. The light is then focused by a second lens onto an aperture for depth discrimination and then detected by a highly sensitive photo multiplier tube (PMT).

The output current of the PMT is converted into a voltage read by an analog to digital converter (ADC) and the processed data is passed back to the computer as the fluorescent intensity level of the sample point, or picture element (pixel) currently being scanned. The computer displays the data as an image, as the scan progresses. In addition, the fluorescent intensity level of all samples, representing the expression profile of the sample, is recorded in computer readable format.

If necessary, an experimentally determined correction for “cross talk” (or overlap) between the channels for the two fluors may be made. For any particular hybridization site on the transcript array, a ratio of the emission of the two fluorophores may be calculated. The ratio is independent of the absolute expression level of the cognate gene, but may be useful for genes whose expression is significantly modulated by drug administration, gene deletion or any other tested event.

Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent 0 534858 A1, filed Sep. 24, 1992, by Zabeau et al.), or methods selecting restriction fragments with sites closest to a defined mRNA end (see, e.g., Prashar et al., Proc. Natl. Acad. Sci. USA, Vol. 93, pp. 659-663 (1996)). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see, e.g., Velculescu, Science, Vol. 270, pp. 484-487 (1995)) pathway pattern.

Measurement of Other Aspects

In various embodiments of the present invention, aspects of the biological state other than the transcriptional state, such as the translational state, the activity state or mixed aspects can be measured in order to obtain drug and pathway responses. Details of these embodiments are described in this section.

Translational State Measurements

Expression of the protein encoded by the gene(s) can be detected by a probe which is detectably-labeled, or which can be subsequently-labeled. Generally, the probe is an antibody that recognizes the expressed protein.

As used herein, the term “antibody” includes, but is not limited to, polyclonal antibodies, monoclonal antibodies, humanized or chimeric antibodies and biologically functional antibody fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Camett-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler and Milstein, Nature, Vol. 256, pp. 495-497 (1975); and U.S. Pat. No. 4,376,110. The human B-cell hybridoma technique of Kosbor et al., Immunology Today, Vol. 4, p. 72 (1983); Cole et al., Proc. Natl. Acad. Sci. USA, Vol. 80, pp. 2026-2030 (1983); and the EBV-hybridoma technique, Cole et al., “Monoclonal Antibodies and Cancer Therapy”, Alan R. Liss, Inc., pp. 77-96 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies”, Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies, U.S. Pat. No. 4,946,778; Bird, Science, Vol. 242, pp. 423426 (1988); Huston et al., Proc. Natl. Acad. Sci. USA, Vol. 85, pp. 5879-5883 (1988); and Ward et al., Nature, Vol. 334, pp. 544-546 (1989), can be adapted to produce differentially expressed gene-single chain antibodies. Single chain antibodies are formed by linking the heavy and light chain fragments of the Fv region via an amino acid bridge, resulting in a single chain polypeptide.

More preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016 and 5,770,429.

Antibody fragments, which recognize specific epitopes, may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed, Huse et al., Science, Vol. 246, pp. 1275-1281 (1989), to allow rapid and easy identification of monoclonal Fab fragments with the desired specificity.

The extent to which the known proteins are expressed in the sample is then determined by immunoassay methods that utilize the antibodies described above. Such immunoassay methods include, but are not limited to, dot blotting, western blotting, competitive and noncompetitive protein binding assays, enzyme-linked immunosorbant assays (ELISA), immunohistochemistry, fluorescence activated cell sorting (FACS), and others commonly used and widely-described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen.

Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody must be an antibody that is specific for the protein expressed by the gene of interest.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used.

It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of protein which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope.

Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of the translational state may also be performed according to several additional methods. For example, whole genome monitoring of protein (i.e., the “proteome”, Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well known (see, e.g., Harlow and Lane, “Antibodies: A Laboratory Manual”, Cold Spring Harbor, N.Y. (1988), which is incorporated in its entirety for all purposes). In a one preferred embodiment, monoclonal antibodies are raised against synthetic peptide fragments designed based on genomic sequence of the cell. With such an antibody array, proteins from the cell are contacted to the array, and their binding is assayed with assays known in the art.

Alternatively, proteins can be separated by two-dimensional gel electrophoresis systems Two-dimensional gel electrophoresis is well known in the art and typically involves iso-electric focusing along a first dimension followed by SDS-PAGE electrophoresis along a second dimension (see, e.g., Hames et al., “Gel Electrophoresis of Proteins: A Practical Approach”, IRL Press, NY (1990); Shevchenko et al., Proc. Natl. Acad. Sci. USA, Vol. 93, pp. 1440-1445 (1996); Sagliocco et al., “Yeast”, Vol. 12, pp. 1519-1533 (1996); Lander, Science, Vol. 274, pp. 536-539 (1996)). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

### Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical or physical means appropriate to the particular activity being characterized. Where the activity involves a chemical transformation, the cellular protein can be contacted with the natural substrates, and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

Computer Analysis Systems and Methods of Use

The analytic methods described above are preferably implemented by means of an automated system, such as a computer system. Accordingly, this section describes exemplary computer systems, as well as methods and programs for operating such computer systems, which may be used to perform the methods of this invention.

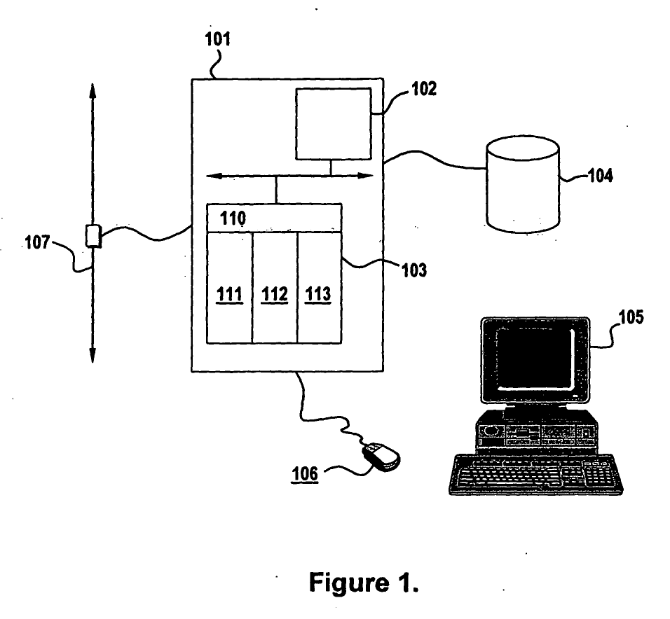

FIG. 1 illustrates an exemplary computer system suitable for implementing the analytic methods of this invention. Computer system 101 comprises internal components linked to external components. The internal components of this computer system include a processor element 102 interconnected with main memory 105. For example, computer system 101 can be an Intel Pentium-based processor of 200 MHz or greater clock rate and with 32 MB or more of main memory.

The external components include mass storage means 104. This mass storage means can be one or more hard disks (which are typically packaged together with the processor and memory). Such hard disks are typically of 1 GB or greater storage capacity.

Other external components may include user interface device 105, which can be a monitor, together with pointing device 106, which can be a “mouse”, or other graphic input devices (not illustrated) and/or a keyboard. A printing device (not shown) can also be attached to the computer system 101.

Typically, computer 101 is also linked to network link 107, which can be part of an Ethernet link to one or more other local computer systems, to one or more remote computer systems, or to one or more wide area communication networks such as the Internet. The network link allows computer system 101 to share data and processing tasks with other computer systems.

Loaded into memory during operation of computer system 101 are several software components which are both standard in the art and special to the instant invention. These software components collectively cause the computer system to function according to the methods of the present invention. These software components are typically stored on mass storage means 104. For example, software component 110 represents an operating system which is responsible for managing computer system 101 and its network interconnections.

The operating system can be, for example, of the Microsoft Windows family, such as Windows95, Windows98, or WindowsNT or may be a Unix operating system such as Sun Solaris.

Software component 110 represents common language and functions conveniently present on computer system 101 to assist programs implementing methods specific to this invention. Many high or low level computer languages can be used to program the analytical methods of this invention. Instruction can be interpreted during run-time, or they is may be interpreted before run time (i.e., compiled) for later execution. Preferred languages include C, C++ and less preferably JAVAO.

Most preferably, the methods of this invention are programmed in mathematical software packages which allow symbolic entry of equations and high-level specification of processing, including algorithms to be used. Such software packages are preferable since they free a user of the need to procedurally program individual equations or algorithms.

Exemplary mathematical software packages which may be used in the computer systems of this invention include Matlab from Mathworks (Natick, Mass.), Mathematica from Wolfram Research (Champaign, Ill.), or S-Plus from Math Soft (Cambridge, Mass.).

Finally, software component 112 represents the analytical methods of the present invention as programmed, e.g., in a procedural language or symbolic package. In a preferred embodiment, the computer system also contains a database 113 of expression profiles.

In certain embodiments software component 110 includes analytic software 112 capable of performing the analysis methods of the present invention. In one embodiment such analytic software would be capable of causing the processor of the computer system to execute steps of (a) receiving data from one or more bioequivalence experiments, preferably from a plurality of bioequivalence experiments, comparing two or more drug formulations or compositions; (b) converting experimental data into expression profiles and into the vector equivalents; (c) determining the similarity between two or more vectors representing expression profiles; and (d) based on this similarity, determining and outputting a measure of the bioequivalence of the two or more drug formulations or compositions being tested.

The data from expression profile experiments may be received, e.g., by a user loading such data into the memory. For example, a user may load such data into the memory from monitor 105 and keyboard 105, or from other computer systems linked by network connection 107, or from storage media 104 including removable storage media such as a CD-ROM or floppy disk.

Preferably, software component 110 includes analytic software components 112 capable of executing the analytic methods of the present invention. In particular, analytic software component 110 preferably comprises components which are capable of comparing vectors represented expression profiles.

Such software an analytic software component preferably causes the processor to execute steps of: (a) receiving one or more expression profiles; (b) calculating the equivalent vector representations; (c) comparing these vectors to determine their degree of similarity, (d) calculating bioequivalence based on this degree of similarity; and (e) outputting this calculated bioequivalence in a user friendly and clinically relevant format.

In a particularly preferred embodiment, the received expression profile data is compared to a database of expression profile data stored in a dynamic database system comprising expression profile data from a plurality of known drug formulations or compositions.

Alternatively, the received expression profile data may loaded by a user, e.g., by any of the above described means.

In a final embodiment, software component 110 also contains analytic software component 112 capable of comparing two or more expression profiles. In particular, analytic software component 112 preferably causes the processor of the computer system to execute steps of: (a) receiving a first expression profile; (b) receiving a second expression profile, (c) converting the received expression profiles into vectors; and (d) calculating the similarity between the first and second expression profile vectors by use of any of the above means.

In certain embodiments, either the first or second expression profile vector received may be inputted by the user. Alternatively, the received expression profile vector may be obtained from a database of expression profile vectors where each is associated with a known standard drug formulation or composition.

Use of the Computer System

In an exemplary implementation, to practice the methods of the present invention, a user first loads expression profile data into the computer system 101. These data can be directly entered by the user from monitor and keyboard 105, or from other computer systems linked by network connection 107, or on removable storage media such as a CD-ROM or floppy disk (not illustrated).

Next the user causes execution of expression profile analysis software 112 which performs the steps of causing the processor to execute steps of: (a) receiving one or more expression profiles, which may come from user input or from the internal database; (b) calculating the equivalent vector representations; (c) comparing these vectors to determine their degree of similarity; (d) calculating bioequivalence based on this degree of similarity; and (e) outputting this calculated bioequivalence in a user friendly and clinically relevant format.

In a less preferable embodiment, the user loads all the expression profile data and the above steps are then performed by the analysis software 112.

The present invention also provides databases of expression profiles for use in bioequivalence determinations according to the methods of this invention. The databases of this invention include expression profiles for a plurality of known and/or standard drug formulations or compositions so that the same database may be used to monitor several different bioequivalence determinations. Preferably, such a database will be in an electronic form that can be loaded into a computer system such as the one illustrated in FIG. 1 and described supra. Such electronic forms include databases loaded into the main memory 104 of a computer system used to implement the methods of this invention, or in the main memory of other computers linked by network connection 107, or on mass storage media 104, or on removable storage media such as a CD-ROM or floppy disk.

In a preferred embodiment, the analytic methods of this invention can be implemented by use of kits for determining the expression profile of a particular drug formulation or composition. Such kits may comprise arrays or microarrays, such as those described above. The microarrays contained in such kits comprise a solid phase, e.g., a surface, to which probes are hybridized or bound at a known location of the solid phase. Preferably, these probes consist of nucleic acids of known, different sequence, with each nucleic acid being capable of hybridizing to an RNA species or to a cDNA species derived therefrom. In particular, the probes contained in the kits of this invention are nucleic acids capable of hybridizing specifically to nucleic acid sequences derived from RNA species which are known to increase or decrease in response to exposure to specific drug formulations or of particular interest to the determination of bioequivalence of the specific drug formulations intended to be monitored by the kit.

The probes contained in the kits of this invention preferably substantially exclude nucleic acids which hybridize to RNA species that are not increased or decreased in response to exposure to specific drug formulations or are otherwise of particular interest to the determination of bioequivalence of drug formulations to be monitored by the kit. In a preferred embodiment, a kit of the invention also contains a database of expression profiles, or the equivalent vector representations, such as the databases described above.

In another preferred embodiment, a kit of the invention further contains expression profile analysis software capable of being loaded into the memory of a computer system such as the one described supra in the subsection, and illustrated in FIG. 1. The expression profile analysis software contained in the kit of this invention, is essentially identical to the expression profile analysis software 112 described above. Such software is capable of executing the analytical steps of the present invention. Preferably, the software causes the processor of the computer system to execute steps of: (a) receiving data from one or more bioequivalence experiments, preferably from a plurality of bioequivalence experiments, comparing two or more drug formulations or compositions; (b) converting experimental data into expression profiles and into the vector equivalents; (c) determining the similarity between two or more vectors representing expression profiles; and (d) based on this similarity, determining and outputting a measure of the bioequivalence of the two or more drug formulations or compositions being tested.

In certain embodiment, either the first or second expression profile or vector or both may be inputted by the user. Alternatively, the received expression profile vector may be obtained from a database of expression profile vectors where each is associated with a known and/or standard drug formulation or composition.

Alternative systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### EXAMPLES

The following examples are presented by way of illustration of the previously described invention and are not limitations of that invention.

### Example 1

### Determination of Bioequivalent Formulations of the Oral Formulation of Pimecrolimus (ASM981)

Pimecrolimus (also called ELIDEL® or ASM981) is an Ascomycin Macrolactam derivative with anti-inflammatory properties. The chemical name of ELIDEL® or ASM981 is 33-Epichloro-33-desoxy-ascomycin. ASM981 has been successfully tested in animal models and in clinical trials against inflammatory skin diseases using a topical formulation.

More recently, topical ASM981 or pimecrolimus, in the form of 1% cream, has been used with great success in the treatment of psoriasis, atopic dermatitis and contact dermatitis. In addition an oral formulation has been used to treat patients with moderate to severe plaque psoriasis and has also proved highly successful.

ASM981 belongs to the macrolatam derivative family and as with other members of this family it binds to macrophilin 12 and inhibits calcineurin. ASM981 is able to block the activation of both T cells and mast cells and the synthesis and release of inflammatory cytokines.

Psoriasis is the prototypical papulosquamous condition, characterized by well-demarcated erythematous papules and plaques with silvery scale. Psoriasis is, at least in part, genetically determined. It is usually a chronic condition of epidermal proliferation and dermal inflammation. Psoriasis begins most commonly in early adult life, but it may begin at any age. The disease may remain localized to just a few areas or may cause continuous generalized disease, occasionally resulting in total-body erythema and scale, termed erythroderma.

The precise pathogenesis of psoriasis is unknown, but it is known to be a chronic inflammatory disease of immune origin. During the development of the disease leukocytes are activated and lymphocytes infiltrate the skin, which is the target organ of the disease.

There is an increased prevalence of psoriasis in individuals with human leukocyte antigens (HLAs) HLA BW17, B13 and BW37 and 30% of patients have a family history of disease suggesting the influence of a genetic predisposition. The basic alteration represents an accelerated cell cycle in an increased number of dividing epidermal basal cells, culminating in rapid epidermal cell proliferation. Cellular turnover is increased up to seven-fold, and the transit time from the basal layer to the top of the stratum corneum is 34 days rather than the usual 28. This rapid turnover of keratinocytes alters keratinization, resulting in thickened epidermis (seen as papules and plaques) and parakeratotic stratum corneum (silver scales). T lymphocytes play a role, but the exact mechanism underlying this benign proliferative reaction is unknown (see Cecil Textbook of Medicine, 211 Ed., Goldman, Bennett, Eds., W.B. Saunders Company, Philadelphia, Pa. (2000) (especially pp. 2281-2281)).
Structure of “33epichloro-33-desoxy-ascomvcin” 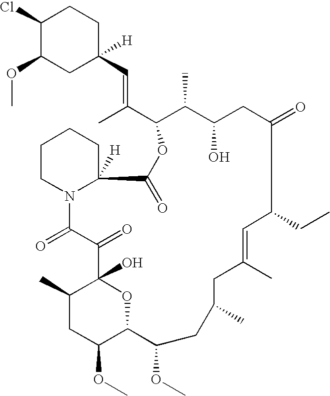
Pimecrolimus or ELIDEL®

A clinical study was initiated with a standardized oral formulation of ASM981 (pimecrolimus) to treat psoriatic patients. The purpose of this study was both to investigate the nature of the alteration in gene expression profiles caused by the administration of a standardised oral formulation of pimecrolimus and to produce the necessary data to allow the bioequivalence of other pharmaceutically equivalent oral formulations of pimecrolimus to be tested.

The oral formulation of pimecrolimus (ASM981) used in this study was formulated as shown below. The “solid dispersion” formulation of pimecrolimus consists of ASM981 20%, poloxamer188 10% and HPMC 70% and the manufacture of this formulation is as disclosed in U.S. Pat. Nos. 6,004,973 and 6,197,781, both of which are hereby incorporated by reference in their entirety and for all purposes. In addition, pimecrolimus and combinations and oral formulations are disclosed in U.S. Pat. Nos. 6,197,781 and 6,004,973; and in EP 0 427 680 B1, WO 01/90110 A1 and WO 97/03654, all of which are hereby incorporated by reference for all purposes.

The oral formulation (formulation A) used in this study were 20 mg tablets consisting of:

| Component | mg/dosis |
|---|---|
| ASM981 solid dispersion 20% | 100.0 |
| Lactose, anhydrous | 98.75 |
| Polyvinylpolypyrrolidon XL | 50.0 |
| Magnesium stearate | 1.25 |
| Total tablet weight | 250.0 |

Placebo tablets matching the 20 mg active tablets were formulated as follows:

| Component | mg/dosis |
|---|---|
| Hydroxypropylmethyl-cellulose 3 CPS (Shin-Etsu) | 45.0 |
| Poloxamer 188 | 5.0 |
| Lactose, anhydrous | 148.75 |
| Polyvinylpolypyrrolidon XL | 50.0 |
| Magnesium stearate | 1.25 |
| Total tablet weight | 250.0 |

The dose used was 60 mg/day, given as 30 mg twice a day (b.i.d.). To make this dose the 20 mg tablets were cut in half using a special cutter (Tablettenteiler NR.96) so as to obtain 30 mg of ASM981 or a matched amount of placebo.

ASM981 was evaluated as treatment for psoriasis using an oral dose of 60 mg (30 mg b.i.d) during a 4 weeks clinical study. The oral formulation used was a highly standardized oral dosage form that could be produced in large amounts and with a highly uniform composition, this was oral formulation A.

The cohort included 10 patients; 8 received oral formulation A and 2 received a placebo. Blood samples were collected before the first administration to establish the gene expression baseline and on days 13 or 14 after treatment with ASM981 or placebo and submitted to gene expression analysis in order to gain insights into the mechanisms of action of the compound.

The clinical data demonstrate a reduction of psoriasis in the 8 ASM981-treated patients. The RNA from each of the blood samples was extracted and tested on individual DNA microarrays, thus allowing, for each patient, a comparison of gene expression before and after ASM981 treatment. One aspect of this study was to establish a profile of gene modulation participating to our understanding of the clinical regression of psoriasis observed in treated patients and/or potential side effects. Genes that were consistently modulated, i.e., change greater then a factor of two, were sorted in functional categories. Table 4 lists all the identified genes with their symbol, Unigene cluster number, protein accession number and the GenBank accession number for the sequence used to design the Affymetrix probes.

A consistent genomic profile of ASM981 (about 160 genes) was identified. The compound was shown to down regulate the expression of genes belonging to the Macrolactam target pathway (Macrophilin 12), cellular activation and proliferation (Histone2, Histone 3.3, cyclin D2) among blood cells, and to strongly down regulate the expression of inflammatory mediators (Leukotriene A4 hydrolase, Prostaglandin endoperoxide synthase). ASM981 efficacy as clinically observed may also be due to the dramatic down regulation of genes necessary for chemotaxis and cellular migration at the site of inflammation, including LFA-1, P-Selectin ligand and L-Selectin.

It was discovered during this study, that treatment with this oral formulation of ASM981 also markedly down regulated several genes known to be involved in asthma physiopathology, these included RANTES, GALECTIN-3 and Thromboxane A2 receptor. This discovery suggested that ASM981 may also be beneficial for this indication as well as other inflammatory diseases. It was also discovered in this study that no consistent pattern of alteration of genes involved in any known adverse side effect was observed.

The genomic analysis of the peripheral blood from 8 psoriatic patients before and after treatment showed a consistent genomic “signature” of ASM981. This signature constituted the characteristic gene expression profile of this specific oral formulation and is shown in Table 13. This table shows the number and name of each gene and the ratio or fold change in gene expression product, comparing pre and post drug exposure, this constitutes the gene expression profile for this specific oral formulation of ASM981, i.e., formulation A. This gene expression profile can be compared by the methods of this invention to the gene expression profile of an unknown or new but pharmaceutically equivalent formulation of ASM981 to determine bioequivalence and therefore the possibility of substitution.

The data also showed that ASM981 acts via multiple targets and mainly by anti-inflammatory activity. The markers identified by gene expression profiling following ASM981 treatment suggest other interesting indication for the compound including asthma and inflammatory bowel disease.

The extraction of the RNA from the samples, the preparation of the target, the hybridization and the analysis of the arrays, were performed according to the methods and protocols recommended by Affymetrix. Total RNA was extracted from the blood samples (lymphocytes) collected on days 0 and 14 using TRIZOL™ (Life Technologies), and purified through RNEASY™ columns (Qiagen) as needed. Good quality total RNA (5 mg) was used to synthesize double-stranded cDNA using the SUPERSCRIPT CHOICE SYSTEM™ (Life Technologies). The cDNA was then in vitro transcribed (MEGASCRIPT™ T7 Kit, Ambion) to form biotin-labeled cRNA. The quality of the cRNA was checked on a test array according to the recommendation of Affymetrix. Next, 12-15 mg of labeled cRNA were hybridized to the probe arrays for 16 hours at 45° C. Arrays were then washed according to the EukGE-WS2 protocol (Affymetrix), and stained with 10 mg/mL of streptavidin-phycoerythrin conjugate (Molecular Probes). The signal was antibody amplified with 2 mg/mL acetylated BSA (Life Technologies), 100 mM MES, 1 M [Na+], 0.05% Tween 20, 0.005% Antiofoam (Sigma), 0.1 mg/mL goat IgG and 0.5 mg/mL biotinylated antibody and re-stained with the streptavidin solution. After washing, the arrays were scanned twice with the GENE ARRAY® scanner (Affymetrix).

The data were analyzed comparing, for each individual patient, the gene expression levels before and after treatment. Only genes that were consistently modulated in at least 5 patients were considered as relevant. These variations were also compared with the gene expression changes observed in the placebo treated patients. During analysis, one chip (patient 44 before treatment) appeared to have very few correctly hybridized genes, and to introduce a bias into the analysis. This pair was excluded to establish average fold change.

A large majority of genes are down regulated. A list of 160 modulated genes was established, comprising 140 (88%) down-regulated genes and 20 up-regulated genes (see Appendices 1-12 and Table 13).

A total of 163 genes consistently modulated were divided into functional categories as follows:
Immunosuppression related genes (13 genes)
   Genes related to FK506/ASM981 immunosuppressive pathway (4 genes)
   HLA and antigen presentation related genes (9 genes)
Leukocytes trafficking related genes (24 genes)
   Chemotaxis and migration (13 genes)
   Cytoskeleton and cellular deformation related genes (11 genes)
Inflammation related genes (12 genes
Cellular activation and proliferation (105 genes)
   Ribosomal protein (52 genes)
   Protein synthesis and intracellular trafficking (14 genes)
   Proliferation related genes (10 genes)
   DNA-/RNA-binding proteins and transcription factors (9 genes)
   Intracellular signalling (7 genes)
   Cellular metabolism (12 genes)
Genes coding for protein without a known function (10 genes)

The complete list and description of modulated genes is presented in Tables 1-13.

Immunosuppression Related Genes

Genes Related to FK506/ASM981 Immunosuppressive Pathway (Table 1)

The four listed genes, macrophilin 12 (FKBP 12), Neuragranin, Calmodulin and Calcium Modulating cyclophilin ligand belong to an identified immunosuppressive pathway.

HLA and Antigen Presentation Related Genes (Table 2)

Invariant parts of the HLA system are common to each patient and are found among consistently down regulated genes. These down regulation affects class I like and class II genes and to a lesser extend class I genes.

Leukocytes Trafficking Related Genes

During an immune or auto immune response, the migration of effector lymphocytes to the location of the exogenous or endogenous target is a critical step for a successful reaction.

Cellular migrations are also critical during any inflammatory reaction.

The migration is usually initiated by a chemotactic signal attracting cells at the target location. Leukocytes are able to recognise activated endothelia and adhere to them.

ASM981 treatment seems to down regulate the expression of genes belonging to each part of this sequence of events.

Chemotaxis and Migration (Table 3)

The down regulation of LFA1 affects both component of the heterodimer. This adhesion molecule is of particular interest and has been designated as a target for treatment of skin inflammatory disease (FSC proposal RD-1999-03563). MIC-2 is thought to regulate LFA-1 expression.

RANTES is a major chemotactic molecule and as been identified as key mediator in some immune-mediated inflammatory reactions.

P-Selectin ligand and L-Selectin are enabling lymphocytes to reach the inflammation site.

Cytoskeleton and Cellular Deformation Related Genes (Table 4)

Inflammation Related Genes (Table 5)

Cellular Activation and Proliferatfon (Tables 6-12)

Ribosomal Proteins (Table 6)
Protein Synthesis and Intracellular Trafficking (Table 7)
Proliferation Related Genes (Table 8)
DNA-/RNA-Binding Proteins and Transcription Factors (Table 9)
Intracellular Signalling (Table 10)
   Cellular Metabolism (Table 11)
   Genes Coding for Protein Without a Known Function (Table 12)
   Markers for T Lymphocytes

Since ASM981 was tested for its immunosuppressive capacity, thus markers for T lymphocyte down regulation drew a particular attention. The behavior of CD8 and granzyme B and A, as well as CD3 chain were observed. The results are listed in Table 13. ASM981 does not affect the proportion of T cell expressing CD3 in the blood. It even seems that CD8+ T lymphocytes are slightly more represented in the blood after treatment and could account for the increase of granzyme A and B. CD4 could not be monitored due to a lack of specific hybridization with the CD4 probes on the chips.

Four different genes can be linked to the Macrolactam immunosuppressive pathway. ASM981, like FK506, is a calcineurin inhibitor and shares with it the ability to bind macrophilin as FKBP12, although with a 3-fold lower affinity than FK506 (see Bochelen et al., J. Pharmacol. Exp. Ther., Vol. 288, No. 2, pp. 653-659 (1999)). FKBP12 is strongly down regulated at the level of RNA synthesis, whereas ASM981 binds to FKBP12 protein. The sequestration of FKBP12 by ASM981 was supposed to induce an increase of the mRNA expression as a compensatory mechanism, however, the expression pattern obtained suggests that ASM981 is also blocking the expression of FKBP12 at the transcriptional level, thus preventing the replenishment of cellular free FKBP12 stores. CAMLG is a natural ligand for one of the cyclbsporin A ligand, this down-regulation may indicates that ASM981 targets different members among the immunophilin family of proteins.

Calmodulin and neuragranin are two calcium-binding proteins participating to the storage release and intracellular distribution of calcium, thus also able to interfere with signal transduction pathways.

The ASM981 profile shows a decrease of cellular activation as seen with the down regulation of cellular proliferation markers, protein synthesis, cellular metabolism and ribosomal proteins. This effect can be a consequence of the calcineurin inhibition, inducing a blockade of activation and proliferation. Ribosomal proteins expression is deeply affected suggesting that some cells may have stopped proliferating.

ASM981 also affects the expression of HLA class I like genes (e.g., HALF) and HLA class II (Ii invariant chain, necessary for the proper intracellular processing of class II complex).

HLA class lb genes are expressed by a large variety of cell types whereas class II genes are expressed by antigen-presenting cells like macrophages, B cells and dendritic cells. This type of down regulation may impair the maintenance of an immune or autoimmune reaction.

The development of inflammation requires the migration of cells at the site. ASM981 impairs multiple steps of the migration as chemoattraction, leukocytes rolling, firm adhesion to the endothelium and extravazation.

The down regulation of chemoattractant RANTES could severely affect the ability of cells to reach the site of inflammation. The decrease in RANTES expression is of particular interest since it perfectly matches the reported in vitro effect of ASM981 namely, impaired inflammatory mediators release, decrease of the IgE promoter activity, decrease of T cell activation and proliferation (see Bochelen et al., supra).

L-selectin and P-selectin ligand, key molecules in the leukocyte rolling steps are also down regulated. P-selectin ligand can bind various lectins and participate to cell/endothelium interactions. This protein is post transcriptionally modified and one of the modification variants is known as CLA (Cutaneous Lymphocyte Antigen). CLA is thought to be responsible for targeting lymphocytes to the skin. The down regulation of CIA would also impair lymphocyte migration at the site of lesions extravazation.

The ASM981 treatment also down-regulated the expression of inflammation related genes.

Both chains, alpha and beta, of the LFA-1 protein are down regulated. LFA-1 is a key molecule in the infiltration and T cell activation processes. An antibody against LFA-1 has been proven to be effective in phase II studies in psoriatic patients (see Grassberger et al., Br. J. Dermatol., Vol. 141, No. 2, pp. 264273 (1999)). Inhibitors of LFA-1 potently inhibit allergic contact dermatitis in a mouse model. Here, ASM981 may block a critical point of the inflammation process.

ASM981 is also down regulating cytoskeleton proteins, thus possibly impairing cellular major enzymes as prostaglandin endoperoxide synthase and leukotriene A4 hydrolase are down-regulated; Kallistatin, an inhibitor of kinins generation is up-regulated. ASM981 seems to efficiently block the inflammatory potential of circulating cells.

Taken together data showed that ASM981 is able to down regulate:
The macrophilin regulation pathway
Cellular activation and proliferation
HLA expression
Cellular migration at the inflammation site
Inflammation

No obvious toxic effect could be observed in the expression profile. No gene linked to a possible adverse effect or to a toxicity pathway was found to be regulated.

The observed gene expression profile does not allow the determining of the cellular subsets that are affected by the treatment. This down regulations could affect every type of leukocyte or only some of them. However, the proportion of T cells seems to remain constant as indicated by the unchanged CD3 expression level (CD3 only states the presence of T cells and does not indicate their activation state). The slight increase in CD8 and granzyme expressions could be a sign of a differential effect on different lymphocyte subsets, that needs to be confirmed by clinical data and/or the analysis of other samples. It could also indicate that some compartments of the immune system are not disabled. ASM981 has been targeted as an anti-inflammatory compound to treat skin diseases.

ASM981 has been proven to be effective in animal models of contact dermatitis by topical application and oral route (see Grassberger et al., supra). Clinical trials with ASM981 have been conducted using topical formulation either to treat atopic dermatitis or psoriasis, leading to the clinical regression (see Gottlieb et al., J. Am. Acad. Dermatol., Vol. 42, No. 3, pp. 428-435 (2000); Meingassner et al., Br. J. Dermatol., Vol. 137, No. 4 (1997). The anti-inflammatory properties of ASM981 have also been successfully tested in another experimental systems including reperfusion after ischemia in the brain (see Bochelen et al., supra).

As discussed above, the strong impact on RANTES, adhesion molecules and inflammatory proteins such as Thromboxane A2 receptor (responsible for bronchoconstriction in asthma) indicates that this oral formulation of ASM981 may also be effective in the treatment of asthma.

Asthma is a clinical syndrome whose precise etiology is not known. However, inflammation is known to play a large role in the disease. Clinically asthma is characterized by three distinct components: (1) recurrent episodes of airway obstruction that resolve spontaneously or as a result of treatment; (2) an exaggerated bronchoconstrictor response to stimuli that have little or no effect in non-asthmatic subjects, a phenomenon known as airway hyperresponsiveness; and (3) inflammation of the airways as defined by a variety of criteria.

Asthma is an extremely common disorder, affecting men and women equally; approximately 5% of the adult population of the United States has signs and symptoms consistent with a diagnosis of asthma. Although most cases begin before the age of 25 years, asthma may develop at any time throughout life. The worldwide prevalence of asthma has increased more than 30% since the late 1970s. The greatest increases in asthma prevalence have occurred in countries that have recently adopted an “industrialized” lifestyle. In addition, the burden of severe asthma has fallen disproportionately on socioeconomically disadvantaged dwellers in the inner city. The reasons for the overall increase in asthma prevalence or the disproportionate fraction of cases in the inner city are not known.

Asthma is among the most common reasons to seek medical treatment; in the United States, asthma is responsible for about 15 million annual outpatient visits to physicians and for nearly 2 million annual inpatient hospital days of treatment. The yearly direct and indirect costs of asthma care are more than six billion dollars, with more than 80% of these costs attributable to direct expenditures on medical care encounters or asthma medications.

In addition, and of great interest, is the finding that the large panel of inflammatory related genes that are down regulated strongly suggest that inflammatory bowel diseases (IBD) may be a potential indication for ASM981 in addition to asthma and possibly other diseases which involve inflammation. IBD, including ulcerative colitis and Crohn's disease, are chronic inflammatory diseases of the gastrointestinal tract. (For information on asthma and IBD, see Cecil Textbook of Medicine, 21^{st} Ed., Goldman and Bennett, Eds., W.B. Saunders Company, Philadelphia, Pa. (2000)).

Determination of Bioequivalence

As discussed above the expression profile produced by the measurement of alterations in a plurality of mRNAs as a result of the administration of a standardized oral formulation of ASM981, i.e., formulation A, can be used to determine the bioequivalence of a different but pharmaceutically equivalent oral formulation of ASM981.

The goal of this comparison would be to determine whether or not a different, but pharmaceutically equivalent formulation of ASM981 could be substituted for the standard formulation A without significant changes in the clinical effects of the resulting treatment. This comparison would be done in the following fashion. First, the formulation to be compared to formulation A would be administered to analogous subjects under circumstances which would allow complete pharmaceutical equivalence to be maintained. This would require that the two formulations, A and B, contain the same active ingredients (chemically identical active drug substance or substances) and are identical in strength or concentration, dosage form and route of administration and that the subjects be “analogous”.

Under the above circumstances, the pharmaceutical effects of the two formulations will be identical if and only if these two-pharmaceutically equivalent drug formulations are also “bioequivalent”, meaning that the rates and extents of bioavailability of the active ingredient(s) in the two drug formulations are not significantly different under these otherwise identical test conditions. The alterations in the gene expression profile in the subjects receiving formulation B would then be determined in precisely the same manner as it was for formulation A, described above. The result of this determination would be the production of an expression profile for formulation B containing all or most of the 163 elements (altered mRNA species) as the expression profile for formulation A shown in Table 13.

The expression profile for formulation A, already determined, could be stored in the memory of one of the embodiments of the computer systems disclosed or could be inputted by the user at the time of the comparison process. The expression profile for formulation B would then be inputted into the computer system by any method and the software would then perform the actions required to determine the degree of bioequivalence of the two formulations.

These actions could comprise; causing execution of expression profile analysis software which performs the steps of causing the computers processor to execute steps of: (a) receiving one or more expression profiles, which may come from user input or from the internal database; (b) calculating the equivalent vector representations; (c) comparing these vectors to determine their degree of similarityl; (d) calculating bioequivalence based on this degree of similarity; and (e) outputting this calculated bioequivalence in a user friendly and clinically relevant format.

The step of comparing the vectors to determine their degree of similarity could be accomplished by any of the mathematical methods disclosed above. For example, the computer could calculate the generalized cosine angle between the two vector representations of the multi element expression profile and thereby provide a correlation coefficient with a value of from −1 to +1. A correlation coefficient of +1 would indicate that the two vectors were identical while a coefficient of 0 would indicate no relationship between the two vectors. A coefficient of −1 would indicate a perfect negative association and therefore negative coefficients are, in this example, not relevant. The sensitivity, with regard to the underlying biology, of this technique would be extremely high so that even small deviations from a correlation coefficient of +1 would indicate significant and clinically relevant variations in bioequivalence between formulations A and B.

For the purposes of this example, a correlation coefficient of less than 0.98 would indicate a clinically relevant lack of bioequivalence between the two formulation and this would dictate that formulation B could not be substituted for formulation A without significant and possibly dangerous changes in the efficacy or side effects of the resulting treatment.

### TABLES

TABLE 1
Down regulation of 4 genes belonging to the FK506/ASM981 pathway

| | | Mean fold |
|---|---|---|
| Gene | Function | variation |
| Macrophilin 12 FKBP | Target for the immunosuppressive | −66.2 |
| | drug (8) | |
| | Regulated at the RNA level | |
| Calmodulin | Calcium binding protein | −28.8 |
| | Regulation of Ca intracellular | |
| | signaling | |
| | T cell activation pathway (9) | |
| CAMLG (calcium | T cell activation pathway | −5.2 |
| modulating cyclophilin | downstream of the TCR and | |
| ligand) | upstream of calcineurin | |
| | Binds to cyclophilin B (like CSA) | |
| | Activates NF-AT and NF-IL2A (10) | |
| NRGN Neuragranin | Calmodulin binding protein | −3.1 |
| | Storage and release of calmodulin | |
| | and calcium (11) | |

TABLE 2
HLA and antigen presentation related genes

| | | Mean fold |
|---|---|---|
| Gene | Function | variation |
| li Invariant chain, | Association of HLA class II molecule | −46.4 |
| CD74 | Protection of the peptide presenting | |
| | groove | |
| HLA-DR | HLA class II molecule | −3.2 |
| HLA-E | Class I like molecule recognized by NK | −23.4/−4.2 |
| (2 sequences) | lymphocytes (12) | |
| HLA-F | Class I like molecule: Induced by IFN | −10.2 |
| | gamma (13) | |
| HLA-I C | Class I like molecule (14) | −2.9 |
| HLA-I A 26 | Class I molecule | −4.1 |
| LAMP-1 | Lysosomal membrane glycoprotein | −19.1 |
| LAPT5 | Marker for hematopoietic cells | −12.5 |
| Lysosomal | Lysosomes: antigen processing for | |
| associated | HLA class II | |
| protein | | |
| 4-1BB ligand | Expressed by B lymphocytes, | +2.3 |
| | macrophages, and Dendritic cells. | |
| | Very efficient CD8+ T cell | |
| | co-stimulation (15) | |

TABLE 3
Down regulation of chemotaxis or migration related proteins

| Gene | Function | Mean fold variation |
|---|---|---|
| L-selectin | Leukocyte rolling. Migration. Absence of L-selectin | −46.6 |
| | impairs migration (16). | |
| LFA1a | Migration at the inflammation site. Target for | −16.1/−8.3 |
| LFA1b | psoriasis treatment and transplantation tolerance | |
| | (17, 18). Antibodies against LFA-1 induce a | |
| | diminution of psoriatic lesions. Abolishes contact | |
| | sensitivity (19) Co blockade with CD28 prevents | |
| | GVHD in mice (20). | |
| MIC-2 CD99 | CD99 unknown ligand Adhesion and aggregation of | −65 |
| | lymphocytes | |
| | Regulates LFA-1/CAM-1 adhesion of lymphocytes | |
| | (21) | |
| RANTES | Binds CCR-1, -3, -4, -5 Chemoattractant for | −5.3 |
| | lymphocytes, monocytes, eosinophils, basophils | |
| | T cell proliferation activation (22), Th1 differentiation. | |
| | Release of inflammatory mediators (23). | |
| | Activates IgE synthesis (24). Expressed in psoriatic | |
| | lesions (25) and eosinophils from atopic asthmatics | |
| | Present in BAL and serum in lung inflammation | |
| | and asthma (26, 27) | |
| | In vitro study. ASM981: Diminution of T cell | |
| | proliferation. Prevents releasing of pro-inflammatory | |
| | mediators. Reduces activity of the IgE promoter | |
| | RANTES antagonist blocks hyper reactivity and | |
| | eosinophils recruitment in a mouse model of | |
| | asthma (28) | |
| PF-4 | Platelet Factor Activation 4 | −5.2 |
| | Leukocyte recruitment, histamine release. | |
| | Marker for inflammatory bowel disease (29) | |
| Selectin P | Tethering and rolling of neutrophils and T | −17.1 |
| ligand | lymphocytes on endothelium. | |
| SELPLG | Interacts with P and L-selectins (30, 31) | |
| | One isoform of SELPLG is CLA (Cutaneous | |
| | Lymphocyte Associated antigen) (32) | |
| Platelet | Adhesion (33). Generation of cytoplasmic processes. | −26.6/−5.2 |
| glycoprotein | lb; Von Willebrand factor binding site | |
| lb/llb | lb.: Mac-1 counter receptor trans platelet migration | |
| | (34) | |
| Proteoglycan | Adhesion. Chemokines storage and release (35) | +49 |
| core protein | | |
| GPR-9 CCR9 | Chemokine receptor. Up regulated. | +3.4 |
| | Binding of SLC, ELC and TECK (36) | |
| | ELC, SLC T cells and dendritic cells trafficking, lymph | |
| | node homing and recirculation | |
| DARC | Chemokines receptor | +7 |
| | Chemokine sequestration (37) Up regulated. | |
| Butyrophilin | Adhesion molecule | −10.6 |
| BTF5 | | |

TABLE 4
Cytoskeleton and cellular deformation related genes

| | | Mean fold |
|---|---|---|
| Gene | Function | variation |
| Vimentin | Cytoskeletal protein | −7 |
| CAPL/S100A4 | Calcium binding protein: Co localizes | −37.8 |
| | with myosin | |
| BRCA2 | Actin related protein. Organization of | −6.5 |
| | lamellipodia | |
| Non muscle-type | Mobilization of actin. Cell mobility | −2.9 |
| cofilin | | |
| Gamma actin | Cytoskeletal protein | −3.4 |
| Myosin light | Cell motility. Membrane blebbing. | −15.6 |
| chain non | Transepithelial migration (neutrophils): | |
| muscle type | Interacts with actin. | |
| Myosin regulatory | Cellular migration | −8.5 |
| light chain | | |
| Laminin receptor | Interaction with extracellular matrix | −7.3 |
| Cystatin C | Inhibition of Cathepsin B responsible | −25 |
| | for matrix degradation | |
| Cystatin A | Protease inhibitor for matrix degrading | −2.4 |
| | enzymes | |
| Thymosin beta | Actin binding protein | −7.5 |

TABLE 5
Inflammation related genes

| Gene | Function | Mean fold variation |
|---|---|---|
| Ferritin | Elevated during acute and chronic | −4.4 |
| | inflammation induced by prostaglandin A1 | |
| | (38) | |
| | H-Kininogen binding (39) | |
| Leukotriene A4 | Responsible for the synthesis of LTB4 (40) | −8 |
| hydrolase | | |
| FC-epsilon binding | Fc Epsilon binding and mast cell activation | −21 |
| protein Galectin 3 | (release of inflammatory mediators) (41) | |
| | Allergic states: Asthma (42) | |
| Thromboxane A2 | Smooth muscle contraction | −9.3 |
| receptor | Bronchoconstriction in asthma (43) | |
| | Blockade suppresses MCP-1 expression by | |
| | activated vascular endothelial cells (44) | |
| Prostaglandin | First step of Leukotrienes and | −45.9 |
| endoperoxide synthase 1 | thromboxanes synthesis | |
| | Target for aspirin (45) | |
| Kallistatin | Kallikrein binding protein Inhibits Kallikrein | +3.3 |
| | kininogenase (46) | |
| Prosaposin | Precursor protein for 4 sphingolipidases | −56.1 |
| | activators. Role of biological detergent | |
| | lifting substrates out the membranes plane | |
| | (47) | |
| Farnesyl transferase | Inflammation marker. Inhibition of Ft inhibits | −9 |
| | corneal inflammation (48) | |
| TRMP2 Apolipoprotein J | Inflammation marker. Injury induced (49) | −6.8 |
| Glutathione peroxidase | GSH metabolism: Correlation of eosinophil | −5.4 |
| | count and glutathione peroxidase in the | |
| | blood of asthmatic patients (50) | |
| Nuclear factor Nf-IL6 | Up regulates the activity of IL1 b core | −6.8 |
| | promoter (51) | |
| Transaldolase | Glutathione metabolism (52) | −3.1 |

TABLE 6
Ribosomal proteins

| | Mean | | Mean fold |
|---|---|---|---|
| Small subunit (24) | fold variation | Large subunit (26) | variation |
| S 2 | −2.9 | L 4 | −6.4 |
| S 3 | −15.9 | L 7 | −7.3 |
| S3a | −3.5 | L 9 | −.1 |
| S 6 | −5 | L 10a | −4.8 |
| S 7 | −6.4 | L 12 | −4.8 |
| S 8 | −4.2 | L 11 | −4.8 |
| S9 | −3.4 | L 13a | −4.8 |
| S 10 | −109.5 | L 17 | −33.5 |
| S 11 | −84 | L 18 | −75.6 |
| S 12 | −4 | L 18a | −4.6 |
| S 13 | −3.7 | L 19 | −21.6 |
| S 14 | −5.8 | L 27a | −7.5 |
| S 16 | −5.5 | L 27 | −5 |
| S 17 | −4.3 | L 28 | −5.5 |
| S 18 | −4.4 | L 29 | −4.6 |
| S 19 | −21.6 | L 30 | −10.6 |
| S 20 | −4.5 | L 31 | −5.8 |
| S 21 | −2.4 | L 32 | −3.1 |
| S 24 | −4 | L 34 | −3.6 |
| S 24 | −4 | L 35a | −37.4 |
| S 25 | −3.5 | L 37 | −3.2 |
| S 27a | −4 | L 37a | −4 |
| S 27 | −7.3 | L 38 | −4.5 |
| S 28 | −4 | L 39 | −3 |
| S 29 | −2.7 | L 41 | −2.7 |
| | | L 44L | −2.8 |

Ribosomal Phosphoproteins

| | |
|---|---|
| Phosphoprotein P1 | −3.8 |
| Phosphoprotein P0 | −127 |

TABLE 7
Protein synthesis and intracellular trafficking

| | | Mean |
|---|---|---|
| Gene | Function | fold variation |
| Dynein | Intracellular trafficking | −26.7 |
| Prefoldin 5 | Intracellular trafficking. Protein | −24.7 |
| | folding | |
| PABP | Poly A binding | −5.3 |
| | Translation control. | |
| Nascent polypeptide | Ribosomes binding to the | −13.6 |
| associated complex | reticulum membrane | |
| NAC | | |
| Spermidine/spermine | Protein synthesis | −21.6 |
| N-acetyl transferase | | |
| Ornithine | Protein synthesis | −134.6 |
| decarboxylase | | |
| Clathrin associated | Vesicle trafficking | −22.7 |
| protein | | |
| Poly C binding protein | Target for HPV16 induced | −16 |
| | translational inhibition | |
| Calnexin IP90 | Chaperon | −1.94 |
| Interferon inducible | | −6.7 |
| protein p27 | | |
| Interferon inducible | | −2.9 |
| protein 1-8 D | | |
| IRF5 | Possible down regulation of | +4.3 |
| | interferon gamma induced | |
| | genes | |
| Amyloid A4 precursor | Amyloid protein. Alzheimer | −16.1 |
| | disease | |
| ETF-1gamma | Eukaryotic | −6.3 |
| | translation factor | |

TABLE 8
Proliferation related genes

| | | Mean fold |
|---|---|---|
| Gene | Function | variation |
| Scaffold attachment | Chromatin structure | −7.1 |
| protein | | |
| Histone H3.3 | Chromatin structure | −6.6 |
| Histone H2 | Chromatin structure | −42.6 |
| Zinc finger protein | Antiproliferative properties. Up | +3.2 |
| ZNF141 | regulated | |
| Cyclin D2 | Expressed in B and T cells. Progression | −16.6 |
| | in G1 phase | |
| Nucleosome | Assembly of nucleosomes | −7.7 |
| assembly protein | | |
| Prothymosin alpha | Proliferation marker | −71.7 |
| | Interaction with histone H1.Chromatine | |
| | unfolding | |
| Cyclin D3 | T and B cells Progression G1 phase | −7.2 |
| CSF-1 | Granulocytes, macrophage and | +3.1 |
| | monocytes activation | |

TABLE 9
DNA-/RNA-binding proteins and transcription factors

| Gene | Function | Mean fold variation |
|---|---|---|
| EF-1 | Elongation factor 1 delta | −12.4 |
| HMG-17 | DNA binding protein non histone | −15.5 |
| MTf1 | Mitochondrial transcription factor | +3.2 |
| TF lia | Transcription factor | −16.4 |
| Wilms tumor related protein | Transcription factor | −4 |
| TAX REB 67 | DNA binding protein: Enhances | −22.6 |
| | transcription of gadd 153 during stress | |
| | response | |
| ALu | RNA binding protein | −2.9 |
| Single stranded DNA | DNA binding | −8.5 |
| binding | | |
| Small nuclear | RNA binding | −2 |
| ribonucleoprotein | | |

TABLE 10
Intracellular signalling

| | | Mean fold |
|---|---|---|
| Gene | Function | variation |
| Serine Threonine | Signal transduction | +5.1 |
| phosphatase | | |
| Tyrosine phosphatase | Signal transduction | −5.7 |
| (huPP1) | | |
| HLH G0S8 | Protein G signaling pathway | −10.8 |
| G gamma 11 | Protein G signaling pathway | −6.2 |
| SABP1 (elk4) | Signaling growth factor | +6.4 |
| | response | |
| BAP 31 | Cell death regulator | −9.2 |
| RSU | Ras inhibitor | −7.2 |
| | Enhances response to | |
| | epidermal growth factor | |

TABLE 11
Cellular metabolism

| Gene | Function | Mean fold variation |
|---|---|---|
| Taurine transporter | Intracellular transport of metabolites | +4.4 |
| Vacuolar H + ATPase | Luminal acidification in the collecting | +1.8 |
| | tube | |
| Glutamine synthase | | −2.6 |
| Lactate dehydrogenase | | −17.1 |
| Cytochrome c oxidase | | −21.1 |
| subunit VIc | | |
| Cytochrome c oxidase | | −15.8 |
| subunit IV | | |
| ADP/ATP translocase | Mitochondrial carrier | −3.7 |
| Mitochondrial ubiquinone | | −5.4 |
| binding protein | | |
| Mitochondrial ubiquinone | | +6.6 |
| binding protein | | |
| Neutrophil cytochrome b | | −34.7 |
| light chain | | |
| Ubiquitin like protein | | −21.5 |
| Thioredoxin binding | Regulation of thioredoxin (thiols | −5.4 |
| protein/Vit. D3 Up regulated | reducing system) | |
| protein | | |
| Pyrroline5 carboxylate | Proline catabolism | +34 |
| reductase | | |

TABLE 12
Genes coding for protein without a known function

| Gene | Function | Mean fold variation |
|---|---|---|
| CDC10 homologue | | −6 |
| Clone × 101 | | +2.2 |
| KIAA0 151 | | +2.5 |
| KIAA0055 | Ubiquitin specific protease? | +3.7 |
| Epididymal secretory protein | | −19.6 |
| Lone HH109 | | +2 |
| pM5 | Similarity with collagenase | −5.7 |
| COX6B | | −13.6 |
| Transiationally controlled | Up regulated in response to PMS or | −3.2 |
| tumor protein | PMA in U937 | |
| B7 protein | | +2.9 |

TABLE 13
Gene expression profile for oral ASM981 formulation A

| | | GenBank | | | | |
|---|---|---|---|---|---|---|
| | | accession | | | | Expression |
| | | number | | | | ratio of |
| | | (used to design) | Unigene | Gene | Protein | mean fold |
| No. | Gene | Affymetrix Probes) | cluser no. | symbol | accession no. | variation |
| 1 | Macrophillin 12 FKBP | M34539 | 752 | FKBP1A | pdb:3FAP | −66.2 |
| 2 | Calmodulin | D45887 | 182278 | CALM2 | sp:P49069 | −28.8 |
| 3 | CAMLG | U18242 | 13572 | CAMLG | sp:P49069 | −5.2 |
| 4 | NRGN Neuragranin | X99078 | 26944 | NRGN | sp:Q92686 | −3.2 |
| 5 | Ii Invariant chain, CD74 | M13560 | NA | IAIG6 | NA | −46.4 |
| 6 | HLA-DR | X00274 | 76807 | HLA-DRA | pdb:IA6A | −3.2 |
| 7 | HLA-E | X56841 | 181392 | HLA-E | pdb:1MHE | −23.4 |
| 8 | HLA-F | X17093 | 110309 | HLA-F | pir:A60384 | −10.2 |
| 9 | HLA-I C | X58538 | 277477 | HLA-C | pir:I37528 | −2.9 |
| 10 | HLA-I A 26 | D32129 | 181244 | HLA-A | pir:I38443 | −4.1 |
| 11 | LAMP-I | J04182 | 150101 | LAMP1 | pir:A31959 | −19.1 |
| 12 | LAPT5 lysosomal-associated | U51240 | 79356 | LAPTM5 | plr:G02476 | −12.5 |
| | protein | | | | | |
| 13 | 4-1BB ligand | U03398 | 1524 | TNFSF9 | pir:I38427 | +2.3 |
| 14 | L-selectin | M25280 | 82848 | SELL | pir:JL0104 | −46.6 |
| 15 | LFA1a | Y00798 | 174103 | ITGAL | pdb:1CQP | −16.1 |
| 16 | LFAIb | M15395 | 83968 | ITGB2 | pir:IJHULM | −8.3 |
| 17 | MIC-2 CD99 | M16279 | 177543 | MIC2 | pir:A60592 | −65 |
| 18 | RANTES | M21121 | 241392 | SCYA5 | pdb:1RTN | −5.3 |
| 19 | PF-4 | M26167 | 72933 | PF4V1 | pir:PFHU4A | −5.2 |
| 20 | Selectin P ligand | U25956 | 79283 | SELP1G | sp:Q14242 | −17.1 |
| 21 | Human platelet glycoprotein | M34344 | 785 | ITGA2B | pir:I57461 | −5.2 |
| | IIb (GPIIb) | | | | | |
| 22 | Proteoglycan core protein | X17042 | 1908 | PRG1 | pir:A28058 | +49 |
| 23 | GPR-9 CCR9 | U45982 | 225948 | CCR9 | sp:P51686 | +3.4 |
| 24 | DARC | X85785 | 183 | FY | sp:Q16570 | +7 |
| 25 | Butyrophilin BTF5 | U90552 | 284283 | BTN3A1 | NA | −10.6 |
| 26 | Vimentin | Z19554 | 297753 | VIM | sp:P08670 | −7 |
| 27 | CAPLIS100A4 | M80563 | 81256 | S100A4 | pirA48219 | −37.8 |
| 28 | BRCA2 | U50523 | 83583 | ARPC2 | sp:O15l44 | −6.5 |
| 29 | Non muscle-type cofihin | X95404 | 180370 | CFL1 | PIR:S12632 | −2.9 |
| 30 | Gamma actin | M19283 | 14376 | ACTG1 | pir:JC5818 | −3.4 |
| 31 | Myosin light chain non | Hg28515-HT4023 | NA | NACA | NA | −15.6 |
| | muscle type | | | | | |
| 32 | Myosin regulatory light chain | X54304 | 233938 | MLCB | pir:MOHULP | −8.5 |
| 33 | Laminin receptor | M14199 | 181357 | LAMR1 | pir:A31233 | −7.3 |
| 34 | Cystatin C | M27891 | 135084 | CST3 | plr:UDHU | −25 |
| 35 | Cystatin A | D88422 | 621 | CSTA | pir:UDHUS | −2.4 |
| 36 | Thyrnosin beta | S54005 | 76293 | TMSB10 | PID:g339697 | −7.5 |
| 37 | Ferritin | M11147 | 111334 | FTL | pir:FRHUL | −4.4 |
| 38 | Leukotriene A4 hydrolase | J03459 | 81118 | LTA4H | pir:S65947 | −8 |
| 39 | FC-epsilon binding protein | M57710 | 621 | LGALS3 | sp:P17931 | −21 |
| | Galectin 3 | | | | | |
| 40 | Thromboxane A2 receptor | D38081 | 89887 | TBXA2R | pir:A56194 | −9.3 |
| 41 | Prostaglandin endoperoxide | M59979 | 88474 | PTGS1 | pir:JH0259 | −45.9 |
| | synthase 1 | | | | | |
| 42 | Prosaposin | J0377 | 78575 | PSAP | prf:1504251A | −56.1 |
| 43 | Famesyl transferase | L10413 | 138381 | FNTA | pir:A47659 | −9 |
| 44 | TRMP2 | M63379 | NA | TRMP2 | NA | −8.8 |
| 45 | Glutathione peroxidase | Y00433 | 76686 | GPXI | sp:P07203 | −5.4 |
| 46 | Nuclear factor Nf-1L6 | X52560 | 99029 | CEBPB | sp:PI7676 | −6.8 |
| 47 | Transaldolase | L19437 | 77290 | TALDOl | pdb:1F05 | −3.1 |
| 48 | S 2 | X17206 | 182426 | RPS2 | NA | −2.9 |
| 49 | S 3 | X55715 | 252259 | RPS3 | sp:P23396 | −15.9 |
| 50 | S 3a | M84711 | 77039 | RPS3A | pir:JC4662 | −3.5 |
| 51 | S 6 | M77232 | 241507 | RPS6 | pir:R3HU6 | −5 |
| 52 | S 7 | Z25749 | 301547 | RPS7 | pir:JC4388 | −6.4 |
| 53 | S 8 | X67247 | 151604 | RPS8 | sp:P09058 | −4.2 |
| 54 | S 9 | U14971 | 180920 | RPS9 | pir:S55917 | −3.4 |
| 55 | S 10 | U14972 | 76230 | RPS10 | pir:S55918 | −109.5 |
| 56 | S 11 | X06617 | 182740 | RPS11 | pir:R3HU11 | −84 |
| 57 | S 12 | HG613-HT613 | 285405 | RPS12 | pir:R3HU12 | −4 |
| 58 | S 13 | HG821-HT821 | 165590 | RPS13 | pir:S34109 | −3.7 |
| 59 | S 14 | M13934-cds2 | 244621 | RPS14 | par:A25220 | −5.8 |
| 60 | S 16 | M60854 | 80617 | RPS16 | NA | −5.5 |
| 61 | S 17 | M18000 | 5174 | RPS17 | pir:R4HU17 | −4.3 |
| 62 | S 18 | X69150 | 275865 | RPS18 | pir:S30393 | −4.4 |
| 63 | S 19 | M81757 | 298262 | RPS19 | sp:P39019 | −21.6 |
| 64 | S 20 | HD1800-HT1823 | 8102 | RPS20 | pr:S33710 | −4.5 |
| 65 | S 21 | L04483 | 1948 | RPS21 | NA | −2.4 |
| 66 | S 24 | M31520-mal-s | 180450 | RPS24 | pir:JH0213 | -4 |
| 67 | S 25 | M64716 | 113029 | RPS25 | pir:JQ1347 | −3.5 |
| 68 | S 27 | HG3214-HT3391 | 195453 | RPS27 | SP:P42677 | −7.3 |
| 69 | S 28 (?23) | D14530 | 3463 | RPS23 | sp:P39028 | −4 |
| 70 | S 29 | U14973 | 539 | RPS29 | NA | −2.7 |
| 71 | L 4 | D23860 | 286 | RPL4 | pir:T09551 | −6.4 |
| 72 | L 7 | X57959 | 153 | RPL7 | pir:S30212 | −7.3 |
| 73 | L 9 | U09953 | 157850 | RPL9 | pir:S42108 | −0.1 |
| 74 | L 10a | U12404 | 252574 | RPL10a | NA | −4.8 |
| 75 | L 12 | L06505 | 182979 | RPL12 | pir:S35531 | −4.8 |
| 76 | L 11 | X79234 | 179943 | RPL11 | sp:P39026 | −4.8 |
| 77 | L 13a | X56932 | 119122 | RPL13a | sp:P40429 | −4.8 |
| 78 | L 18 | L11568 | 75458 | RPL18 | sp:Q07020 | −75.6 |
| 79 | L 18a | X80822 | 163593 | RPL118a | sp:Q02543 | −4.6 |
| 80 | L 19 | X63527 | 25273 | RPL19 | pir:A487992 | −21.6 |
| 81 | L 17 | X53777 | 82202 | RPL17 | pir:R5HU22 | −33.5 |
| 82 | L 27a | U14968 | 76064 | RPL27A | pir:S55914 | −7.5 |
| 83 | L 27 | L19527 | 111611 | RPL27A | pir:S43505 | −5 |
| 84 | L 28 | U14989 | 4437 | RPL28 | pir:S55915 | −5.5 |
| 85 | L 29 | Z49148 | 183698 | RPL29 | pir:S65784 | −4.6 |
| 86 | L 30 | HG311-HT311/ | 111222 | RPL30 | pir:S45004 | −10.6 |
| | | HG2873-HT3017 | | | | |
| 87 | L 31 | X15940 | 184014 | RPL31 | pir:R5HU31 | −5.8 |
| 88 | L 32 | X03342 | 169793 | RPL32 | pir:R5HU32 | −3.1 |
| 89 | L 34 | L38941 | 250895 | RPL34 | pir:I68524 | −3.6 |
| 90 | L 35a | X52966 | 287361 | RPL35a | pir:R5HU35 | −37.4 |
| 91 | L 37 | HG3364-HT3541 | 179779 | RPL37 | sp:P02403 | −3.2 |
| 92 | L 37a | L06499 | 5566 | RPL37a | PID:g7020475 | −4 |
| 93 | L 38 | Z26876 | 2017 | RPL38 | NA | −4.5 |
| 94 | L 39 | D79205 | 300141 | RPL39 | NA | −3 |
| 95 | L 41 | Z12962 | 324408 | RPL41 | sp:Q16465 | −2.7 |
| 96 | L 44L | U78027 | 178391 | RPL44 | sp:P09896 | −2.8 |
| 97 | Phosphoprotein P1 | M17886 | 177592 | RPLP1 | pir:R6HUP1 | −3.8 |
| 98 | Phosphoprotein P0 | M17885 | 73742 | RPLP0 | pir:R5HUP0 | −127 |
| 99 | Dynein | U32944 | 5120 | PIN | pdb:1CMI | −26.7 |
| 100 | Prefoldin 5 | D89667 | 288858 | PFDN5 | NA | 24.7 |
| 101 | PABP | U68105 | | PABP | NA | −5.3 |
| 102 | Nascent polypeptide | X80909 | 32916 | NACA | pir:S49326 | −13.6 |
| | associated complex NAC | | | | | |
| 103 | Spermidine/sperrnine | U40369 | 28491 | SAT | pir:JH0783 | −21.6 |
| | N-acetyl transferase | | | | | |
| 104 | Omithine decarboxylase | D78361 | NA | NA | NA | −134.6 |
| 105 | Clathrin associated protein | X97074 | Hs.119591 | AP2S1 | sp:P53680 | −22.7 |
| 106 | Poly C binding protein | Z29505 | Hs.2853 | PCBP1 | pir:S58529 | −16 |
| 107 | Calnexin IP90 | L10284 | Hs.155560 | CANX | sp:P27824 | −1.94 |
| 108 | Interferon inducible protein | J04164 | Hs.148360 | IFITM1 | pir:A31454 | -6.7 |
| | p27 | | | | | |
| 109 | Interferon inducible protein | X57351 | Hs.174195 | IFITM2 | pir:S17183 | −2.9 |
| | 1-8 D | | | | | |
| 110 | IRF5 | US1157 | Hs.54434 | IRF5 | pir:G02474 | +4.3 |
| 111 | Amyloid A4 precursor | Y00264 | Hs.177486 | APP | pdb:1AML | −16.1 |
| 112 | ETF-1gamma | M55409 | Hs.2186 | EEF1G | sp:P26641 | −6.3 |
| 113 | Scaffold attachment protein | D13413 | Hs.103804 | HNRPU | sp:Q00839 | −7.1 |
| 114 | Histone H3.3 | M11353 | Hs.181307 | H3F3A | sp:P0835l | −6.6 |
| 115 | Histone H2 | U90551 | Hs.28777 | H2AFL | sp:Q93077 | −42.6 |
| 116 | Zinc finger protein ZNF141 | L15309 | Hs.193677 | ZNF141 | prf:1923299A | +3.2 |
| 117 | Cyclin D2 | D13639 | Hs.75586 | CCND2 | pir:A42822 | −16.6 |
| 118 | Nucleosome assembly protein | M86667 | Hs.179662 | NAP1L1 | pir:S40510 | −7.7 |
| 119 | Prothymosin alpha | M14483 | Hs.250655 | PTMA | sp:P06454 | −71.7 |
| 120 | Famesyt transferase alpha | L10413 | Hs.138381 | FNTA | pir:A47659 | −9.0 |
| | subunit | | | | | |
| 121 | Cyclin D3 | M92287 | Hs.83173 | CCND3 | pir:B42822 | −7.2 |
| 122 | CSF-1 | X05825 | Hs.173894 | CSF1 | sp:P09603 | +3.1 |
| 123 | EF-1 | Z21507 | Hs.223241 | EEF1D | sp:P29692 | −12.4 |
| 124 | HMG-17 | X13546 | Hs.181163 | HMG17 | pir:S03700 | −15.5 |
| 125 | MTf1 | X64269 | Hs.239379 | TFAM | NA | +3.2 |
| 126 | TF lia | HG4312-HT4582 | NA | NA | NA | −16.4 |
| 127 | Wilms tumor related protein | HG3549-HT3751 | NA | NA | NA | −4 |
| 128 | TAX REB 67 | D90209 | Hs.181243 | ATF4 | sp:P18848 | −22.6 |
| 129 | Alu | U07857 | Hs.180394 | SRP14 | pdb:1E8O | −2.9 |
| 130 | Single-stranded DNA binding | M94556 | Hs.923 | SSBP | pdb:3ULL | −8.5 |
| 131 | Small nuclear | J04615 | Hs.48375 | SNRPN | pir:A33270 | −2 |
| | ribonucleoprotein | | | | | |
| 132 | Senne threonine | M65254 | Hs.108705 | PPP2R1B | sp:P30154 | +5.1 |
| | phosphatase | | | | | |
| 133 | Tyrosine phosphatase | U14603 | Hs.82911 | PTP4A2 | pir:I68523 | −5.7 |
| | (huPP1) | | | | | |
| 134 | HLH G0S8 | L13391 | Hs.78944 | RGS2 | sp:P41220 | −10.8 |
| 135 | G gamma 11 | U31384 | Hs.83381 | GNG11 | pir:I39159 | −6.2 |
| 136 | SABP1 (elk4) | M85164 | Hs.169241 | ELK4 | pir:A53012 | +6.4 |
| 137 | BAP 31 | X81817 | Hs.291904 | DXS1357E | pir:JC2386 | −9.2 |
| 138 | RSU | L12535 | Hs.75551 | RSU1 | sp:Q15404 | −7.2 |
| 139 | Taurine transporter | Z18956 | Hs.1194 | SLC6A6 | pir:S46487 | +4.4 |
| 140 | Vacuolar H + ATPase | Z71460 | Hs.267871 | ATP6N1A | pir:T46449 | +1.8 |
| 141 | Glutamine synthase | X59834 | Hs.170171 | GLUL | sp:P15104 | −2.6 |
| 142 | Lactate dehydrogenase | Xl 3794 | Hs.234489 | LDHB | sp:P07195 | −17.1 |
| 143 | Cytochrome c oxidase | Xl 3238 | Hs.74649 | COX6C | sp:P09669 | −21.1 |
| | subunit VIc | | | | | |
| 144 | Cytochrome c oxidase | U90915 | Hs.113205 | COX4 | prf:1312293A | −15.8 |
| | subunit IV | | | | | |
| 145 | ADP/ATP translocase | J03592 | Hs.164280 | SLC25A6 | pir:S03894 | −3.7 |
| 146 | Mitochondrial ubiquinone- | M26730 | NA | NA | NA | −5.4 |
| | binding protein | | | | | |
| 147 | Neutrophil cytochrome b | M21186 | Hs.68877 | CYBA | pir:A28201 | −34.7 |
| | light chain | | | | | |
| 148 | Ubiquitin-like protein | J03589 | Hs.76480 | UBL4 | pir:A31084 | −21.5 |
| 149 | Thioredoxin-binding protein/ | S73591 | Hs.179526 | VDUP1 | prf:2019235A | −5.4 |
| | Vitamin D3 up-regulated | | | | | |
| | protein | | | | | |
| 150 | Pyrroline5 carboxylate | M77836 | Hs.79217 | PYCR1 | sp:P32322 | +34 |
| | reductase | | | | | |
| 151 | CDC10 homologue | S72008 | Hs.184326 | CDC10 | pir:JC2352 | −6 |
| 152 | Clone x 101 (putative | Z47038 | NA | MAP1A | NA | +2.2 |
| | microtubute-associated | | | | | |
| | protein lA) | | | | | |
| 153 | KIAA0 151 | D63485 | Hs.321045 | IKKE | PID:g6012176 | +2.5 |
| 154 | KIAA0055 | D29956 | Hs.152818 | USP8 | sp:P40818 | +3.7 |
| 155 | Epididynisl secretory protein | X67698 | Hs.119529 | HEl | sp:Q15668 | −19.6 |
| 156 | Lone HH109 | D23873 | Hs.84883 | K1AA0864 | NA | +2 |
| 157 | pM5 | X57398 | Hs.227823 | PM5 | sp:Q15155 | −5.7 |
| 158 | COX6B | AC0022115 | Hs.174031 | COX6B | pir:OGHU6B | −13.6 |
| 159 | Translationally-controlled | X16084 | Hs.279860 | TPT1 | pir:S06590 | −3.2 |
| | tumor protein | | | | | |
| 160 | B7 protein | U72508 | Hs.155586 | B7 | NA | +2.9 |

### REFERENCES CITED

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art.

Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. A method for determining the degree of bioequivalence of two pharmaceutically equivalent drug formulations when contacting analogous biological samples comprising:
(a) obtaining a first expression profile by measuring abundances of a first plurality of cellular constituents in a biological sample contacted with a first drug formulation or composition;
(b) obtaining a second expression profile by measuring abundances of second plurality of cellular constituents in an analogous biological sample contacted with a pharmaceutically equivalent second drug formulation or composition;
(c) comparing the thus obtained expression profiles; and
(d) determining the degree of bioequivalence between the two drug formulations or compositions by comparing the degree of similarity between the expression profiles.

2. The method of claim 1, wherein the said biological sample is a mammal.

3. The method of claim 1, wherein the said biological sample is a human.

4. The method of claim 1, wherein the said biological sample is a cell culture.

5. The method of claim 1, wherein the said biological sample is a yeast.

6. The method of claim 1, wherein the said biological sample is a non-mammalian animal.

7. The method of claim 1, wherein the step of comparing the expression profiles is performed by converting the expression profiles into vectors and comparing the vectors by use of a similarity metric.

8. The method of claim 7, wherein the similarity metric is the generalized cosine angle.

9. The method of claim 7, wherein the step of determining the degree of bioequivalence is performed by determining the correlation coefficient from the similarity metric.

10. The method of claim 1, wherein said first plurality of cellular constituents and said second plurality of cellular constituents comprise the abundances of a plurality of RNA species present in said cells.

11. The method of claim 10, wherein said first plurality of abundances of RNA species constitutes abundances of the majority of RNA species known to be increased or decreased in a cell in response to administration of a first known drug formulation.

12. The method of claim 10, wherein said abundances are measured by a method comprising contacting a gene transcript array with RNA from said cells, or with cDNA derived therefrom, wherein a gene transcript array comprises a surface with attached nucleic acids or nucleic acid mimics, said nucleic acids or nucleic acid mimics capable of hybridizing with said plurality of RNA species, or with cDNA derived therefrom.

13. The method of claim 1, wherein said first plurality of cellular constituents and said second plurality of cellular constituents comprise abundances of a plurality of protein species present in said cells.

14. The method of claim 13, wherein said abundances are measured by a method comprising; contacting an antibody array with proteins from said cells, wherein said antibody array comprises a surface with attached antibodies, said antibodies capable of binding with said plurality of protein species.

15. The method of claim 13, wherein said abundances are measured by a method comprising performing two-dimensional electrophoresis of proteins from said cells.

16. The method of claim 1, wherein said cellular constituents comprise activities of a plurality of protein species present in said cell type.

17. The method of claim 1, wherein the first drug formulation is the ASM981 oral formulation called formulation A.

18. The method of claim 1, wherein the first expression profile is the expression profile of the ASM981 oral formulation called formulation A and shown in Table 13.

19. A method for selecting a bioequivalent replacement drug formulation for use in treating a patient in need of such drug treatment comprising:
(a) obtaining a first expression profile by measuring abundances of a first plurality cellular constituents in a cell or cells from a subject administered a first known drug formulation;
(b) obtaining a second expression profile by measuring abundances of a second plurality of cellular constituents in a cell or cells of an analogous subject or subjects administered a pharmaceutically equivalent therapy with said replacement drug formulation;
(c) comparing the thus obtained expression profiles;
(d) determining the degree of bioequivalence between the two drug formulations by comparing the degree of similarity between the expression profiles;
(e) assessing, from the degree of bioequivalence determined in (d), whether or not the said replacement drug formulation will produce a clinical result in patients that is sufficiently similar to the clinical result of the first known or standard drug formulation to allow the substitution of the first drug formulation with the second drug formulation; and
(f) selecting, for use in treating a patient, in need of such drug treatment, the said replacement drug formulation if the assessment in (e) indicates that the substitution can be made.

20. The method of claim 19, wherein said subject is a mammal.

21. The method of claim 19, wherein said subject is a human.

22. The method of claim 19, wherein the step of comparing the expression profiles is performed by converting the expression profiles into vectors and comparing the vectors by use of a similarity metric.

23. The method of claim 22, wherein the similarity metric is the generalized cosine angle.

24. The method of claim 22 or 23, wherein the step of determining the degree of bioequivalence is performed by determining the correlation coefficient from the similarity metric.

25. The method of claim 19, wherein said first plurality of cellular constituents and said second plurality of cellular constituents comprise the abundances of a plurality of RNA species present in said cells.

26. The method of claim 25, wherein said first plurality of abundances of RNA species constitutes abundances of the majority of RNA species known to be increased or decreased in a cell in response to administration of a first known drug formulation.

27. The method of claim 25, wherein said abundances are measured by a method comprising contacting a gene transcript array with RNA from said cells, or with cDNA derived therefrom, wherein a gene transcript array comprises a surface with attached nucleic acids or nucleic acid mimics, said nucleic acids or nucleic acid mimics capable of hybridizing with said plurality of RNA species, or with cDNA derived therefrom.

28. The method of claim 19, wherein said first plurality of cellular constituents and said second plurality of cellular constituents comprise abundances of a plurality of protein species present in said cells.

29. The method of claim 28, wherein said abundances are measured by a method comprising; contacting an antibody array with proteins from said cells, wherein said antibody array comprises a surface with attached antibodies, said antibodies capable of binding with said plurality of protein species.

30. The method of claim 28, wherein said abundances are measured by a method comprising performing two-dimensional electrophoresis of proteins from said cells.

31. The method of claim 19, wherein said cellular constituents comprise activities of a plurality of protein species present in said cell type.

32. The method of claim 19, wherein the first drug formulation is the ASM981 oral formulation called formulation A.

33. The method of claim 19, wherein the first expression profile is the expression profile of the ASM981 oral formulation called formulation A and shown in Table 13.

34. A method for treating a patient, in need of treatment with a first known drug formulation, with a second drug formulation, comprising:
(a) determining the degree of bioequivalence between a first known drug formulation or composition and a second different, but pharmaceutically equivalent, drug formulation;
(b) assessing, from the degree of bioequivalence determined in (i), whether or not the second drug formulation will produce a clinical result in patients that is sufficiently similar to the clinical result of the first drug formulation to allow the substitution of the first drug formulation or composition with the second drug formulation; and
(c) treating a patient, in need of drug treatment with the first known drug formulation, by administration of the second drug formulation if the assessment in (b) indicates that a substitution can be made.

35. The method of claim 34, wherein the step of determining the degree of bioequivalence is performed by the method of claim 1.

36. The method of claim 34, wherein the step of assessing is performed by comparing the determined correlation coefficient with the correlation coefficients of pairs of drug formulations known to be sufficiently similar to allow the substitution of the first drug formulation or composition with the second drug formulation or composition and if the determined correlation coefficient is no greater that the correlation coefficient of the known pairs than the substitution is allowed.

37. The method of claim 34, wherein the correlation coefficient must be greater than 0.90 for the assessment to indicate that a substitution can be made.

38. The method of claim 37, wherein the correlation coefficient must be greater than 0.95 for the assessment to indicate that a substitution can be made.

39. The method of claim 38, wherein the correlation coefficient must be greater than 0.98 for the assessment to indicate that a substitution can be made.

40. The method of claim 39, wherein the correlation coefficient must be greater than 0.99 for the assessment to indicate that a substitution can be made.

41. The method of claim 40, wherein the correlation coefficient must be greater than 0.995 for the assessment to indicate that a substitution can be made.

42. The method of claim 34, wherein the said first known drug formulation is the oral ASM981 formulation called formulation A.

43. The method of claim 34, wherein the first expression profile is the expression profile of the ASM981 oral formulation called formulation A and shown in Table 13.

44. A computer system for determining the bioequivalence of two or more drug formulations administered to analogous subjects, comprising a processor and a memory coupled to said processor, said memory encoding one or more programs, said one or more programs causing said processor to perform a method comprising:
(a) receiving one or more expression profiles, which may come from user input or from the internal database;
(b) calculating the equivalent vector representations;
(c) comparing these vectors to determine their degree of similarity by means of a similarity metric;
(d) calculating the correlation coefficient from the similarity metric; and
(e) determining the degree of bioequivalence of the two or more drug formulations from the correlation coefficient.

45. The computer system of claim 44, wherein said step of comparing vectors to determine their degree of similarity, is achieved by a method comprising comparing the two or more expression profile vectors by means of a similarity metric which is the generalized cosine angle between the two vector representations of the expression profiles being compared.

46. The computer system of claim 44, wherein one of the expression profiles to be compared is contained in the computer memory.

47. The computer system of claim 44, wherein the expression profile contained in memory is the expression profile of the ASM981 oral formulation called formulation A and shown in Table 13.

48. The computer system of claim 44, wherein both the first expression profile and said second expression profile are made available in said memory.

49. The computer system of claim 44, wherein said programs cause said processor to perform step of calculating and comparing the vectors representing the first and second expression profiles.

50. A kit for determining an expression profile resulting from exposure to a drug formulation in a subject, comprising a solid phase containing on its surface a plurality of nucleic acids of known, different sequences, each at a known location on said solid phase, each nucleic acid capable of hybridizing to an RNA species or cDNA species derived therefrom, wherein said RNA species are known to be increased or decreased at different levels of said effect of said therapy, said plurality substantially excluding nucleic acids capable of hybridizing to RNA species that are not so increased or decreased.

51. A kit for determining the bioequivalence of two or more drug formulations in a subject comprising:
(a) a solid phase containing on its surface a plurality of nucleic acids of known, different sequences, each at a known location on said solid phase, each nucleic acid capable of hybridizing to an RNA species or cDNA species derived therefrom, wherein said RNA species are known to be increased or decreased in response to the drug formulations; and
(b) expression profiles, in electronic or written form, of known or standard drug formulations, wherein said expression profiles comprise measured amounts of a plurality of cellular constituents in a cell or ceils of one or more subjects to whom a known or standard drug formulation has been administered.

52. The kit of claim 51, wherein said expression profiles are in electronic form, and wherein said kit further comprises expression profile analysis software on computer readable medium, said software capable of being encoded in a memory of a computer also having a processor, said encoded software causing said processor to perform a method comprising:
(a) receiving an expression profile of a cell or cells of said subject, said expression profile comprising measured abundances of RNA species or cDNA derived therefrom from said cell;
(b) calculating the equivalent vector representations;
(c) comparing these calculated vectors to one or more vectors calculated from expression profiles stored in the computer memory and determining their degree of similarity by means of a similarity metric which is the generalized cosine angle between the two vector representations of the expression profiles being compared;
(d) determining bioequivalence based on the correlation coefficient from the similarity metric; and
(e) outputting this calculated bioequivalence.

53. The kit of claim 51, wherein the expression profile stored in the computer memory is the expression profile of the ASM981 oral formulation called formulation A and shown in Table 13.

54. A database on computer readable medium comprising the expression profiles for one or more drug formulations wherein said database is in electronic form, wherein said expression profiles comprise measurements of a plurality of cellular constituents in a cell or cells of one or more subjects at one or more levels of exposure to a drug formulation.

55. The database of claim 54, wherein the said expression profiles comprise the expression profile of the ASM981 oral formulation called formulation A and shown in Table 13.

56. The database of claim 54, wherein said plurality of cellular constituents comprise abundances of a plurality of RNA species present in said cells.

57. The database of claim 54, wherein said plurality of cellular constituents comprise abundances of a plurality of protein species present in said cells.

58. The database of claim 54, wherein said plurality of cellular constituents comprise measurements of the activities of a plurality of the protein species present in said ceils.

59. A method for comparing one batch of a drug formulation, with a second batch of the same drug formulation for quality control purposes, comprising:
(a) determining the degree of bioequivalence between a first batch of a drug formulation or composition and a second batch of the drug formulation;
(b) assessing, from the degree of bioequivalence determined in (a), whether or not the second batch of drug formulation will produce a clinical result in patients that is sufficiently similar to the clinical result of the first drug formulation to allow the substitution of the first batch of drug formulation or composition with the second batch of drug formulation or composition; and
(c) finding that batch one and batch two are bioequivalent enough to continue in the manufacturing process to produce a final product if the assessment in (b) indicates that the two batches have a sufficient degree of bioequivalence.

60. The method of claim 59, wherein the step of determining the degree of bioequivalence is performed by the method of claim 1.

61. The method of claim 59, wherein the correlation coefficient must be greater than 0.90 for the assessment to indicate that a substitution can be made.

62. The method of claim 61, wherein the correlation coefficient must be greater than 0.95 for the assessment to indicate that a substitution can be made.

63. The method of claim 62, wherein the correlation coefficient must be greater than 0.98 for the assessment to indicate that a substitution can be made.

64. The method of claim 63, wherein the correlation coefficient must be greater than 0.99 for the assessment to indicate that a substitution can be made.

65. The method of claim 64, wherein the correlation coefficient must be greater than 0.995 for the assessment to indicate that a substitution can be made.

66. The method of claim 59 wherein the said first batch of a drug formulation is the oral ASM981 formulation called formulation A.
